(19) United States
(12) Patent Application Publication
(10) Pub. No.: US 2020/0289424 A1
(43) Pub. Date: Sep. 17, 2020

(54) PHARMACEUTICAL COMPOSITIONS COMPRISING DELAYED RELEASE GELLING AGENT COMPOSITIONS

(71) Applicant: Purdue Pharma L.P., Stamford, CT (US)

(72) Inventors: Meet Desai, East Windsor, NJ (US); Haiyong Hugh Huang, Princeton Junction, NJ (US)

(21) Appl. No.: 16/647,927

(22) PCT Filed: Oct. 1, 2018
(86) PCT No.: PCT/US18/53755

§ 371 Date: Mar. 17, 2020

Related U.S. Application Data
(60) Provisional application No. 62/566,989, filed on Oct. 2, 2017.

(51) Int. Cl. A61K 9/50; A61K 31/485

(52) U.S. Cl. CPC A61K 9/5078; A61K 9/0053; A61K 31/485

(57) ABSTRACT

Disclosed herein are immediate release solid oral dosage forms, their methods of preparation and use. The immediate release solid oral dosage form may comprise an active agent composition in an immediate release form, such as an opioid analgesic composition and a gelling agent composition in a delayed release form.

### FIELD OF THE INVENTION

The present invention relates to the field of pharmaceutical dosage forms that are resistant to tampering and abuse. Specifically, the present invention is directed to an immediate release solid oral dosage form that is abuse deterrent, its methods of preparation and use.

### BACKGROUND OF THE INVENTION

Pharmaceutical products are sometimes subject to abuse. For example, a particular dose of opioid analgesic may be more potent when administered parenterally as compared to the same dose administered orally. Some formulations can be tampered with to facilitate illicit use of the opioid therein. A pattern of such abuse of opioid dosage forms may develop if the dosage form is easy to abuse and/or if the abuser likes the high obtained from the abuse, which stimulates the abuser to take the dosage form again.

The use of gelling agents has been contemplated in order to deter the abuse potential of immediate release dosage forms containing a drug susceptible to abuse, such as an opioid analgesic. One form of abuse is via the crushing of a dosage form in order to liberate the drug contained therein for illicit use such as parenteral administration or through absorption across an external mucosal surface. When the crushed dosage form is mixed with a solution, a viscosity is obtained which inhibits the drug from being drawn into a needle, thereby hindering parenteral abuse. Similarly, when the crushed dosage form is applied to a mucosal surface (e.g., the nasal cavity), the composition forms a gel upon contact with mucosal moisture, thereby inhibiting absorption.

One problem to overcome when incorporating a gelling agent into an immediate release dosage form is the extended release characteristics that such an agent may impart to the immediate release dosage form when included in sufficient amounts to inhibit tampering.

There exists a need in the art for an immediate release solid oral dosage form containing an active agent susceptible to abuse, such as an opioid analgesic, that is resistant to parenteral and nasal abuse. More specifically, there exists a need for an immediate release formulation containing a gelling agent that will maintain its abuse deterrence upon tampering, while maintaining the immediate release nature the active agent susceptible to abuse upon proper administration of the formulation.

### OBJECTS AND SUMMARY OF THE INVENTION

It is an object of certain embodiments of the present invention to provide an immediate release solid oral dosage form comprising an active agent susceptible to abuse (e.g., an opioid analgesic), which is tamper-resistant.

It is an object of certain embodiments of the present invention to provide an immediate release solid oral dosage form comprising an active agent susceptible to abuse (e.g., an opioid analgesic), which is subject to less parenteral abuse than other dosage forms.

It is an object of certain embodiments of the present invention to provide an immediate release solid oral dosage form comprising an active agent susceptible to abuse (e.g., an opioid analgesic), which is subject to less intranasal abuse than other dosage forms.

It is a further object of certain embodiments of the present invention to provide an immediate release solid oral dosage form comprising an active agent susceptible to abuse (e.g., an opioid analgesic), which is subject to less diversion than other dosage forms.

It is a further object of certain embodiments of the present invention to treat a disease or condition (e.g., pain) in human patients by administering an immediate release solid oral dosage form as disclosed herein to a patient in need thereof.

It is a further object of certain embodiments of the present invention to provide a method of treating pain in human patients with an immediate release solid oral dosage form comprising an opioid analgesic while reducing the abuse potential of the dosage form.

It is another object of certain embodiments of the present invention to provide a method of preparing an oral dosage form of an active agent susceptible to abuse (e.g., an opioid analgesic) as disclosed herein.

The above objects and others may be achieved by the present invention which in certain embodiments is directed to a solid oral dosage form comprising an immediate release active agent (e.g., an opioid analgesic) composition and a delayed release gelling agent composition, wherein the solid oral dosage form is free of or substantially free of an extended release active agent composition.

In some embodiments, the present invention is directed to an immediate release active agent (e.g., an opioid analgesic) composition and a delayed release gelling agent composition, wherein the solid oral dosage form is free of or substantially free of an extended release active agent composition, wherein the delayed release gelling agent composition comprises a gelling agent and an enteric material. In some embodiments, the enteric material may dissolve at a pH of 5.5 or higher and not dissolve below a pH of 5.5.

In some embodiments, the ratio of the viscosity of a solution obtained from an intact solid oral dosage form at 5 minutes in about 5 ml water at room temperature to the viscosity of a solution obtained from an intact solid oral dosage form at 5 minutes in about 5 ml 0.1N HCl at room temperature is about 10:1 or more.

In some embodiments, the opioid analgesic in the immediate release solid oral dosage forms disclosed herein may be in the form of one or more particles. Each opioid analgesic particle may comprise (i) an inert core coated with the opioid analgesic, or (ii) the opioid analgesic dispersed in a matrix material.

In some embodiments, the delayed release gelling agent composition in the immediate release solid oral dosage forms disclosed herein may be in the form of one or more particles. Each delayed release gelling agent particle may comprise (i) the gelling agent coated with an enteric material, (ii) an inert core coated with a gelling agent and overcoated with the enteric material, or (iii) the gelling agent dispersed in an enteric matrix material.

In some embodiments, the opioid analgesic in the immediate release solid oral dosage forms disclosed herein may be coated on the one or more delayed release gelling agent particles. In other embodiments, the one or more delayed release gelling agent particles in the immediate release solid oral dosage forms disclosed herein may be dispersed in a matrix (e.g., a matrix tablet) comprising an opioid analgesic. In another embodiment, one or more delayed release gelling agent particles and one or more immediate release opioid particles are contained in a pharmaceutically acceptable capsule.

In some embodiments, tampering with the immediate release solid oral dosage forms disclosed herein imparts a viscosity that makes the dosage form unsuitable for parenteral administration.

In some embodiments, the recovery of the opioid analgesic from the immediate release solid oral dosage forms disclosed herein is less than about 40%, less than about 30%, less than about 20%, less than about 10%, less than about 8%, less than about 6%, less than about 4%, or less than about 2% based on a syringeability test whereby a crushed or intact solid oral dosage form is dissolved with 5 ml or 10 ml of solvent with agitation or without agitation at room temperature and the resultant solution is aspirated with a 18, 22, 25, or 27 gauge needle.

Other objects may be achieved by the present invention which in certain embodiments is directed to a process for preparing a solid oral dosage form comprising (i) preparing a delayed release gelling agent composition; (ii) blending the delayed release gelling agent composition with an active agent (e.g., opioid analgesic); and (iii) compressing the blend into a tablet.

In some embodiments, the process for preparing the immediate release solid oral dosage forms disclosed herein comprises enteric coating the gelling agent by a method selected from the group consisting of rotor powder layering, fluid bed granulation, roller compaction, fluid bed coating (Wurster coating), and combination thereof.

In some embodiments, the process for preparing the immediate release solid oral dosage forms disclosed herein may comprise (i) preparing one or more delayed release gelling agent particles; and (ii) coating the one or more delayed release gelling agent particles with an opioid analgesic, wherein the solid oral dosage form comprises an immediate release opioid analgesic composition, and wherein the solid oral dosage form is free of or substantially free of an extended release opioid analgesic composition. In other embodiments, the process for preparing the immediate release solid oral dosage forms disclosed herein may comprise containing one or more delayed release gelling agent particles and one or more immediate release opioid particles in a pharmaceutically acceptable capsule.

In some embodiments, the process for preparing the immediate release solid oral dosage forms disclosed herein may comprise (i) preparing one or more particles; (ii) coating the one or more particles with an opioid analgesic composition; and (iii) blending the one or more particles coated with opioid analgesic composition with a delayed release gelling agent composition, wherein the solid oral dosage form comprises an immediate release opioid analgesic composition, and wherein the solid oral dosage form is free of or substantially free of an extended release opioid analgesic composition.

In some embodiments, the process for preparing the immediate release solid oral dosage forms disclosed herein may comprise (i) preparing one or more delayed release gelling agent particles; and (ii) dispersing an opioid analgesic composition and the one or more delayed release gelling agent particles in a matrix, wherein the solid oral dosage form comprises an immediate release opioid analgesic composition, and wherein the solid oral dosage form is free of or substantially free of an extended release opioid analgesic composition.

In further embodiments, the present invention is directed to a method of treating a disease or condition (e.g., pain) comprising administering to a patient in need thereof an immediate release solid oral dosage form as disclosed herein.

### BRIEF DESCRIPTION OF THE DRAWINGS

The above and other features of the present disclosure, their nature, and various advantages will become more apparent upon consideration of the following detailed description, taken in conjunction with the accompanying drawings, in which:

FIG. 1 presents a process flow diagram for the manufacture of a solid oral dosage form according to a non-limiting embodiment disclosed herein comprising rotor layered and enteric coated pellets.

FIG. 2 presents a process flow diagram for the manufacture of a solid oral dosage form according to a non-limiting embodiment disclosed herein comprising top sprayed granules.

FIG. 3 presents a process flow diagram for the manufacture of a solid oral dosage form according to a non-limiting embodiment disclosed herein comprising roller compacted and bottom sprayed granules.

FIG. 4 presents a chart showing the Active Agent volume aspirated after a syringeability test performed on intact tablets in tap water at room temperature (Example 8).

FIG. 5 presents a chart showing the Active Agent volume aspirated after a syringeability test performed on crushed and intact tablets in tap water at room temperature (Example 8).

FIG. 6 presents a chart showing the percent assay of Active Agent aspirated after a syringeability test performed on intact tablets in tap water at room temperature (Example 8).

FIG. 7 presents a chart showing the percent assay of Active Agent aspirated after a syringeability test performed on crushed vs. intact tablets in tap water at room temperature (Example 8).

FIG. 8 presents the dissolution profiles for tablet formulations disclosed in Table 4 of Example 2, Table 9 of Example 4, and Table 16 of Example 7.

FIG. 9 presents the dissolution profiles for tablet formulations disclosed in Tables 9 and 10 of Example 4.

FIG. 10 presents the dissolution profiles for tablet formulations disclosed in Tables 4 and 5 of Example 2.

FIG. 11 presents the dissolution profiles for tablet formulations disclosed in Tables 16 and 17 of Example 7.

### DETAILED DESCRIPTION OF THE INVENTION

In one aspect, the invention is directed to a solid oral dosage form, which comprises an opioid analgesic composition in an immediate release form and a gelling agent composition in a delayed release form, wherein the solid oral dosage form is free of or substantially free of the opioid analgesic composition in an extended release form.

In certain embodiments, the opioid analgesic is selected from the group consisting of morphine, hydromorphone, hydrocodone, oxycodone, codeine, levorphanol, meperidine, dihydrocodeine, dihydromorphine, oxymorphone, fentanyl, buprenorphine, pharmaceutically acceptable salts thereof, solvates thereof, prodrugs thereof, and mixtures thereof.

In one embodiment, the solid oral dosage form comprises a therapeutically effective amount of the opioid analgesic. In another embodiment, the solid oral dosage form comprises an analgesically effective amount of the opioid analgesic. In certain embodiments, the solid oral dosage form contains about 0.1% to about 80% (w/w), about 0.5% to about 30% (w/w), or about 1% to about 10% (w/w), of the opioid analgesic.

In another embodiment, the solid oral dosage form of the invention releases at least about 80% of the opioid analgesic within 30 minutes, as measured by in-vitro dissolution in a USP Apparatus 1 (#40 mesh basket) in 900 ml 0.1N HCl at room temperature. Under certain circumstances, the solid oral dosage form releases at least about 85%, at least about 90%, or at least about 95% of the opioid analgesic within 30 minutes, as measured by in-vitro dissolution in a USP Apparatus 1 (#40 mesh basket) in 900 ml 0.1N HCl at room temperature.

In a separate embodiment, the solid oral dosage form of the invention releases at least about 80% of the opioid analgesic within 45 minutes, as measured by in-vitro dissolution in a USP Apparatus 1 (#40 mesh basket) in 900 ml 0.1N HCl at room temperature. Under certain circumstances, the solid oral dosage form releases at least about 85%, at least about 90%, or at least about 95% of the opioid analgesic within 45 minutes, as measured by in-vitro dissolution in a USP Apparatus 1 (#40 mesh basket) in 900 ml 0.1N HCl at room temperature.

In another embodiment, the solid oral dosage form of the invention releases at least about 80% of the opioid analgesic within 60 minutes, as measured by in-vitro dissolution in a USP Apparatus 1 (#40 mesh basket) in 900 ml 0.1N HCl at room temperature. Under certain circumstances, the solid oral dosage form releases at least about 85%, at least about 90%, or at least about 95% of the opioid analgesic within 60 minutes, as measured by in-vitro dissolution in a USP Apparatus 1 (#40 mesh basket) in 900 ml 0.1N HCl at room temperature.

In a certain embodiment, the delayed release gelling agent composition in the solid oral dosage form of the invention comprises a gelling agent and an enteric material. The gelling agent can be, for example, starch, starch derivatives, sodium carboxymethylcellulose, methylcellulose, hydroxyethylcellulose, hydroxypropylcellulose, hydroxypropylmethylcellulose, attapulgites, bentonites, dextrins, alginates, carrageenan, gum tragacanth, gum acacia, guar gum, xanthan gum, pectin, gelatin, kaolin, cross linked polyacrylic acid, polyvinylpyrrolidone, polyethylene oxide, or polyvinyl alcohol, or mixtures thereof. In particular, the gelling agent in the solid oral dosage form of the invention is selected from the group consisting of polyethylene oxide, xanthan gum, cross linked polyacrylic acid, polysaccharides, and mixtures thereof. One example provides that the gelling agent comprises a polymer. The polymer may comprise a polysaccharide (e.g., a microbial polysaccharide) and/or an anionic polymer in a neutral pH aqueous solution. One example of the microbial polysaccharide is a xanthan gum. An example of the anionic polymer comprises a polyacrylic acid or a carbomer homopolymer. In one embodiment, the gelling agent of the solid oral dosage form of the invention comprises xanthan gum and carbomer homopolymer.

In certain embodiments, the solid oral dosage form of the invention contains from about 0.1% to about 50%, from about 0.5% to about 20%, or from about 1% to about 10%, gelling agent (w/w).

In certain embodiments, the invention provides a solid oral dosage form, with the viscosity of a solution obtained from an intact solid oral dosage form at 5 minutes in about 0.5 ml to about 10 ml water at room temperature being about 50 cP or more, about 75 cP or more, about 100 cP or more, or about 125 cP or more, wherein the viscosity is measured by a rotational viscometer. In one embodiment, the viscosity of a solution obtained from a tampered solid oral dosage form at 2 minutes in about 0.5 ml to about 10 ml water at room temperature is about 50 cP or more, about 75 cP or more, about 100 cP or more, or about 125 cP or more, wherein the viscosity is measured by a rotational viscometer. In another embodiment, the viscosity of a solution obtained from an intact solid oral dosage form at 5 minutes in about 0.5 ml to about 10 ml of 0.1N HCl at room temperature is about 50 cP or less, about 40 cP or less, about 30 cP or less, about 20 cP or less, about 10 cP or less, about 5 cP or less, or about 2 cP or less, wherein the viscosity is measured by a rotational viscometer. In another embodiment, the viscosity of a solution obtained from a tampered solid oral dosage form at 5 minutes in about 0.5 ml to about 10 ml of 0.1N HCl at room temperature is about 50 cP or less, about 40 cP or less, about 30 cP or less, about 20 cP or less, about 10 cP or less, about 5 cP or less, or about 2 cP or less, wherein the viscosity is measured by a rotational viscometer. In still another embodiment, the viscosity of a solution obtained from an intact solid oral dosage form at 5 minutes in about 0.5 ml to about 10 ml of 0.1N HCl at room temperature is within about 30%, about 20%, about 10%, or about 5% of the viscosity of a solution obtained from a tampered solid oral dosage form at 5 minutes in about 0.5 ml to about 10 ml of 0.1N HCl at room temperature, wherein the viscosity is measured by a rotational viscometer.

In certain embodiments, the invention provides a solid oral dosage form, wherein the ratio of the viscosity of a solution obtained from an intact solid oral dosage form at 5 minutes in about 5 ml water at room temperature to the viscosity of a solution obtained from an intact solid oral dosage form at 5 minutes in about 5 ml 0.1N HCl at room temperature is about 10:1 or more, about 15:1 or more, about 20:1 or more, about 25:1 or more, or about 30:1 or more, wherein the viscosity is measured by a rotational viscometer.

In another embodiment, the invention provides a solid oral dosage form, wherein the opioid analgesic composition is in the form of one or more particles. Each opioid analgesic particle may comprise the opioid analgesic coated on an inert core. Alternatively, each opioid analgesic particle comprises the opioid analgesic dispersed in a matrix material.

In a separate embodiment, the invention provides a solid oral dosage form, wherein the delayed release gelling agent composition is in the form of one or more particles. Each delayed release gelling agent particle, for example, contains (i) the gelling agent coated with the enteric material, (ii) an inert core coated with the gelling agent and overcoated with the enteric material, or (iii) the gelling agent dispersed in matrix material.

One embodiment of the invention provides a solid oral dosage form, in which the one or more opioid analgesic particles and the one or more delayed release gelling agent particles are contained in a pharmaceutically acceptable capsule. In one instance, the one or more opioid analgesic particles and the one or more delayed release gelling agent particles are compressed into a tablet.

One embodiment of the invention provides a solid oral dosage form, in which the opioid analgesic composition is coated on the one or more delayed release gelling agent particles. In another embodiment, the one or more delayed release gelling agent particles are dispersed in a matrix comprising the opioid analgesic composition.

In another embodiment of the solid oral dosage form of the invention, the delayed release gelling agent composition comprises a gelling agent and an enteric material, wherein the enteric material dissolves above a pH of about 5.5. One instance provides that the enteric material does not dissolve below a pH of about 5.5; for example, the enteric material dissolves above a pH of about 5.5 and does not dissolve below a pH of about 5.5.

The enteric material in accordance to the invention includes, such as, a cellulosic material, an acrylic polymer, a methacrylic polymer, and a mixture thereof. For example, the enteric material can be methacrylic acid/methyl methacrylate, methacrylic acid/ethyl acrylate copolymers, methacrylic acid/methyl acrylate/methyl methacrylate copolymers, shellac, hydroxypropyl methylcellulose phthalate, hydroxyl propyl methyl cellulose acetate succinate, hydroxypropyl methyl cellulose trimellitate, cellulose acetate phthalates, polyvinyl acetate phthalates, or mixtures thereof.

When the delayed release gelling agent composition is in the form of one or more particles, each particle may comprise from about 10% to about 30% (w/w), from about 12% to about 25% (w/w), from about 0.1% to about 50% (w/w), from about 1% to about 20% (w/w), or from about 2% to about 15% (w/w), enteric material.

In certain embodiments, the invention provides a solid oral dosage form further comprising an aversive agent. The aversive agent can be, for example, an emetic, an antagonist, a bittering agent, an irritant, or a mixture thereof. The emetics, for example, can be methyl cephaeline, cephaeline, emetine hydrochloride, psychotrine, O-methylpsychotrine, emetamine, ipecamine, hydro-ipecarnine, ipecacunhic acid, ipecac, or mixtures thereof.

The antagonists that can be used in the invention include, such as, naltrexone, naloxone, nalmefene, cyclazacine, levallorphan, pharmaceutically acceptable salts thereof, solvates thereof, prodrugs thereof, and combinations thereof.

The bittering agent that can be used includes, such as, flavor oils, flavoring aromatics, oleoresins, plant extracts, leaf extracts, flower extracts, fruit extracts, sucrose derivatives, chlorosucrose derivatives, quinine sulphate, denatonium benzoate, or mixtures thereof. In one embodiment, the aversive agent is a bittering agent selected from the group consisting of spearmint oil, peppermint oil, eucalyptus oil, oil of nutmeg, allspice, mace, oil of bitter almonds, menthol, and mixtures thereof. In one embodiment, the bittering agent is extracted from a fruit selected from the group consisting of lemon, orange, lime, grapefruit, and mixtures thereof.

The irritant that can be used as an aversive agent include, such as, a surfactant, capsaicin, capsaicin analog, and mixtures thereof. For example, the capsaicin analog can be resiniferatoxin, tinyatoxin, heptanoylisobutylamide, heptanoyl guaiacylamide, an isobutylamide, a guaiacylamide, dihydrocapsaicin, homovanillyl octylester, nonanoyl vanillylamide, or mixtures thereof. The surfactant can be a poloxamer, a sorbitan monoester, a glyceryl monooleate, sodium lauryl sulfate, or combinations thereof.

In certain embodiments, the solid oral dosage form of the invention contains from about 0.1% to about 30% (w/w), or from about 0.5% to about 20% (w/w), or from about 1% to about 10% (w/w), irritant.

Certain embodiments of the invention provide a solid oral dosage form, which further comprises a pharmaceutically acceptable excipient. Such pharmaceutically acceptable excipients can be, for example, plasticizers, colorants, lubricants, fillers, thermal lubricants, antioxidants, buffering agents, disintegrants, binding agents, diluent, glidant, anti-adherants, sweeteners, chelating agents, flavorants, surfactants, solubilizers, stabilizers, hydrophilic polymers, hydrophobic polymers, waxes, lipophilic materials, absorption enhancers, preservative, absorbent, cross-linking agents, bioadhesive polymers, pore formers, osmotic agents, polycarboxylic acids or combinations thereof. The pharmaceutically acceptable excipient can be present at from about 0.1% to about 99% (w/w), or from about 10% to about 80% (w/w), or from about 15% to about 70% (w/w), of the solid oral dosage form.

The filler in accordance with the invention, for example, can be lactose, dextrose, mannitol, microcrystalline cellulose, or a mixture thereof. One embodiment provides that the glidant comprises silicon dioxide. Another embodiment provides that the lubricant comprises magnesium stearate.

Certain embodiments of the solid oral dosage form of the invention contain a delayed release gelling agent composition, which comprise one or more of rotor layered and enteric coated pellets, top sprayed granules, roller compacted pellets, bottom sprayed granules, or combinations thereof.

The solid oral dosage form of the invention can be in the form of a unitary dosage form, or in the form of a plurality of particles, which can be contained in a pharmaceutically acceptable capsule. Also, the solid oral dosage form can be in the form of a tablet.

In accordance with certain embodiments of the invention, the weight ratio of opioid analgesic to gelling agent in the solid oral dosage form is from about 1:30 to about 30:1, or from about 1:15 to about 15:1, or from about 1:10 to about 10:1, or from about 1:8 to about 8:1, or from about 1:5 to about 5:1, or from about 1:3 to about 3:1, or from about 1:1.5 to about 1.5:1.

In separate embodiments of the invention, the solid oral dosage form contains a gelling agent and an enteric material, wherein the weight ratio of the gelling agent to the enteric material is from about 1:30 to about 30:1, or from about 1:15 to about 15:1, or from about 1:10 to about 10:1, or from about 1:8 to about 8:1, or from about 1:3 to about 3:1, or from about 1:1.5 to about 1.5:1.

When being subject to a syringeability test during which an intact solid oral dosage form of the invention is dissolved with 5 ml of solvent with agitation at room temperature and the resultant solution is aspirated with a 18, 22, 25, or 27 gauge needle, in certain embodiments of the invention, the recovery of the opioid analgesic is less than about 40%, less than about 30%, less than about 20%, less than about 10%, less than about 8%, less than about 6%, less than about 4%, or less than about 2%. Another embodiment provides that, in a syringeability test during which an intact solid oral dosage form of the invention is dissolved with 5 ml of solvent without agitation at room temperature and the resultant solution is aspirated with a 18, 22, 25, or 27 gauge needle, the recovery of the opioid analgesic is less than about 40%, less than about 30%, less than about 20%, less than about 10%, less than about 8%, less than about 6%, less than about 4%, or less than about 2%.

In another embodiment of the invention, when subject to a syringeability test during which an intact solid oral dosage form of the invention is dissolved with 10 ml of solvent with agitation at room temperature and the resultant solution is aspirated with a 18, 22, 25, or 27 gauge needle, the recovery of the opioid analgesic is less than about 40%, less than about 30%, less than about 20%, less than about 10%, less than about 8%, less than about 6%, less than about 4%, or less than about 2%. Another embodiment provides that after a syringeability test during which an intact solid oral dosage form of the invention is dissolved with 10 ml of solvent without agitation at room temperature and the resultant solution is aspirated with a 18, 22, 25, or 27 gauge needle, the recovery of the opioid analgesic is less than about 40%, less than about 30%, less than about 20%, less than about 10%, less than about 8%, less than about 6%, less than about 4%, or less than about 2%.

Certain embodiments of the invention provide that, in a syringeability test when a crushed solid oral dosage form is dissolved with 5 ml of solvent with agitation at room temperature and the resultant solution is aspirated with a 18, 22, 25, or 27 gauge needle, the recovery of the opioid analgesic is less than about 40%, less than about 30%, less than about 20%, less than about 10%, less than about 8%, less than about 6%, less than about 4%, or less than about 2%.

Another embodiment of the invention provides that, in a syringeability test when a crushed solid oral dosage form is dissolved with 10 ml of solvent with agitation at room temperature and the resultant solution is aspirated with a 18, 22, 25, or 27 gauge needle, the recovery of the opioid analgesic is less than about 40%, less than about 30%, less than about 20%, less than about 10%, less than about 8%, less than about 6%, less than about 4%, or less than about 2%.

A further embodiment of the invention provide that, in a syringeability test when a crushed solid oral dosage form is dissolved with 5 ml of solvent without agitation at room temperature and the resultant solution is aspirated with a 18, 22, 25, or 27 gauge needle, the recovery of the opioid analgesic is less than about 40%, less than about 30%, less than about 20%, less than about 10%, less than about 8%, less than about 6%, less than about 4%, or less than about 2%.

Yet another embodiment of the invention provide that, in a syringeability test when a crushed solid oral dosage form is dissolved with 10 ml of solvent without agitation at room temperature and the resultant solution is aspirated with a 18, 22, 25, or 27 gauge needle, the recovery of the opioid analgesic is less than about 40%, less than about 30%, less than about 20%, less than about 10%, less than about 8%, less than about 6%, less than about 4%, or less than about 2%.

According to certain embodiments of the invention, the viscosity of the solid oral dosage form, when mixed with from about 0.5 ml to about 10 ml of water, makes the opioid analgesic unsuitable for parenteral administration. In other embodiments, the viscosity of the solid oral dosage form of the invention, when mixed with from about 0.5 ml to about 10 ml of water, makes the opioid analgesic unsuitable for intravenous administration.

In certain embodiments of a solid oral dosage form of the invention, the delayed release gelling agent composition includes rotor layered and enteric coated pellets, which comprise from about 5% to about 35% (w/w), or from about 5% to about 15% (w/w), or from about 15% to about 25% (w/w), or from about 25% to about 35% (w/w), enteric material based on total weight of the delayed release gelling agent composition. One example provides that the rotor layered and enteric coated pellets comprise about 10% (w/w) enteric coat based on total weight of the delayed release gelling agent composition. Another example provides that the rotor layered and enteric coated pellets comprise about 30% (w/w) enteric coat based on total weight of the delayed release gelling agent composition.

The invention is also drawn to a solid oral dosage form comprising a delayed release gelling agent composition, which comprises top spray granules, with about 90% to about 100% (specifically, about 95% to about 100%; or more specifically, about 99% to about 100%) of the top spray granules incorporated into the solid oral dosage form retained in one or more of a 100 mesh screen, a 200 mesh screen, and a pan.

In separate embodiments, the invention is drawn to a solid oral dosage form comprising a delayed release gelling agent composition, which comprises top spray granules, with about 20% to about 50% (specifically, about 30% to about 45%) of the top spray granules incorporated into the solid oral dosage form being retained in a 100 mesh screen. One embodiment provides that about 40% of the top spray granules incorporated into the solid oral dosage form are retained in a 100 mesh screen.

In certain embodiments, the invention is drawn to a solid oral dosage form comprising a delayed release gelling agent composition, which comprises top spray granules, with about 20% to about 50% (specifically, about 30% to about 45%) of the top spray granules incorporated into the solid oral dosage form being retained in a 200 mesh screen. One embodiment provides that about 40% of the top spray granules incorporated into the solid oral dosage form are retained in a 200 mesh screen.

Other embodiments of the invention provide that, in the solid oral dosage form, the delayed release gelling agent composition comprises top spray granules, with about 10% to about 30% (or about 15% to about 25%) of the top spray granules incorporated into the solid oral dosage form being retained after sifting through a 200 mesh screen. In one instance, about 20% of the top spray granules incorporated into the solid oral dosage form are retained after sifting through a 200 mesh screen.

The delayed release gelling agent composition in the solid oral dosage form of the invention, may comprise roller compacted pellets having a bulk density ranging from about 0.4 g/ml to about 0.6 g/ml. Alternatively, the roller compacted pellets may have a tapped density ranging from about 0.55 g/ml to about 0.65 g/ml.

The solid oral dosage form of the invention can be in the form of a compressed tablet. Certain embodiments are drawn to a compressed tablet having a hardness of about 2 Kp to about 20 Kp, or of about 5 Kp to about 18 Kp.

In certain embodiments, a solid oral dosage form of the invention has a disintegration time in water ranging from about 10 seconds to about 30 minutes, or has a disintegration time in water ranging from about 1 minute to about 10 minutes, or has a disintegration time in water ranging from about 10 seconds to about 2 minutes.

In other embodiments, a solid oral dosage form of the invention has a disintegration time in SGF ranging from about 10 seconds to about 30 minutes, or from about 1 minute to about 10 minutes, or from about 10 seconds to about 2 minutes.

Another aspect of the invention provides a solid oral dosage form comprising an opioid analgesic composition in an immediate release form and a gelling agent composition in a delayed release form. The gelling agent composition comprises a gelling agent and an enteric material coating the gelling agent, wherein the solid oral dosage form releases at least about 85% of the opioid analgesic within 45 minutes as measured by in-vitro dissolution in a USP Apparatus 1 (#40 mesh basket) in 900 ml 0.1N HCl at room temperature.

A further aspect of the invention is drawn to a solid oral dosage form, which comprises an opioid analgesic composition in an immediate release form and a gelling agent composition in a delayed release form,

wherein the solid oral dosage form is free of or substantially free of an opioid analgesic composition in an extended release form,

wherein the gelling agent composition comprises a gelling agent and an enteric material, and

wherein the enteric material dissolves above a pH of about 5.5 and does not dissolve below a pH of about 5.5.

The invention is also drawn to a solid oral dosage form comprising an opioid analgesic composition in an immediate release form and a gelling agent composition in a delayed release form,

wherein the solid oral dosage form is free of or substantially free of an opioid analgesic composition in an extended release form,

wherein the gelling agent composition comprises rotor layered and enteric coated pellets comprising about 10% (w/w) enteric material based on total weight of the pellets, and

wherein the recovery of the opioid analgesic is less than about 15% based on a syringeability test whereby an intact solid oral dosage form is dissolved with about 5 ml to about 10 ml of tap water without agitation at room temperature and the resultant solution is aspirated with a 18, 22, 25, or 27 gauge needle.

A separate aspect of the invention provides a solid oral dosage form comprising an opioid analgesic composition in an immediate release form and a gelling agent composition in a delayed release form,

wherein the solid oral dosage form is free of or substantially free of an opioid analgesic composition in an extended release form,

wherein the gelling agent composition comprises rotor layered and enteric coated pellets comprising about 10% (w/w) enteric material based on total weight of the pellets, and

wherein the recovery of the opioid analgesic is less than about 5% based on a syringeability test during which an intact solid oral dosage form is dissolved with about 5 ml to about 10 ml of tap water with agitation at room temperature and the resultant solution is aspirated with a 18, 22, 25, or 27 gauge needle.

The invention further includes a solid oral dosage form comprising an opioid analgesic composition in an immediate release form and a gelling agent composition in a delayed release form,

wherein the solid oral dosage form is free of or substantially free of an opioid analgesic composition in an extended release form,

wherein the gelling agent composition comprises rotor layered and enteric coated pellets comprising about 30% (w/w) enteric material based on total weight of the pellets, and

wherein the recovery of the opioid analgesic is less than about 20% based on a syringeability test in which an intact solid oral dosage form is dissolved with about 10 ml of tap water with or without agitation at room temperature and the resultant solution is aspirated with a 18, 22, 25, or 27 gauge needle.

The invention is further drawn to a solid oral dosage form comprising an opioid analgesic composition in an immediate release form and a gelling agent composition in a delayed release form,

wherein the solid oral dosage form is free of or substantially free of an opioid analgesic composition in an extended release form,

wherein the gelling agent composition comprises rotor layered and enteric coated pellets comprising about 30% (w/w) enteric material based on total weight of the pellets, and

wherein the recovery of the opioid analgesic is less than about 10% based on a syringeability test whereby an intact solid oral dosage form is dissolved with about 5 ml of tap water without agitation at room temperature and the resultant solution is aspirated with a 18, 22, 25, or 27 gauge needle.

A further separate aspect of the invention provides a solid oral dosage form comprising an opioid analgesic composition in an immediate release form and a gelling agent composition in a delayed release form,

wherein the solid oral dosage form is free of or substantially free of an opioid analgesic composition in an extended release form,

wherein the gelling agent composition comprises rotor layered and enteric coated pellets comprising about 30% (w/w) enteric material based on total weight of the pellets, and

wherein the recovery of the opioid analgesic is less than about 5% based on a syringeability test whereby an intact solid oral dosage form is dissolved with about 5 ml of tap water with agitation at room temperature and the resultant solution is aspirated with a 18, 22, 25, or 27 gauge needle.

In a further aspect, the invention is drawn to a solid oral dosage form comprising an opioid analgesic composition in an immediate release form and a gelling agent composition in a delayed release form,

wherein the solid oral dosage form is free of or substantially free of an opioid analgesic composition in an extended release form,

wherein the gelling agent composition comprises top spray granules, and

wherein the recovery of the opioid analgesic is less than about 2% based on a syringeability test whereby an intact solid oral dosage form is dissolved with about 5 ml to about 10 ml of tap water with or without agitation at room temperature and the resultant solution is aspirated with a 18, 22, 25, or 27 gauge needle.

Yet in another aspect, the invention provides a solid oral dosage form comprising an opioid analgesic composition in an immediate release form and a gelling agent composition in a delayed release form,

wherein the solid oral dosage form is free of or substantially free of an opioid analgesic composition in an extended release form,

wherein the gelling agent composition comprises roller compacted and bottom sprayed granules,

wherein the recovery of the opioid analgesic is less than about 40%, less than about 30%, less than about 10%, or less than about 8% based on a syringeability test whereby an intact solid oral dosage form is dissolved with about 5 ml to about 10 ml of tap water with or without agitation at room temperature and the resultant solution is aspirated with a 18, 22, 25, or 27 gauge needle.

Further aspects of the invention include a solid oral dosage form comprising an opioid analgesic composition in an immediate release form and a gelling agent composition in a delayed release form,

wherein the solid oral dosage form is free of or substantially free of an opioid analgesic composition in an extended release form,

wherein the gelling agent composition comprises rotor layered and enteric coated pellets comprising about 30% (w/w) enteric material based on total weight of the pellets, and

wherein the recovery of the opioid analgesic is less than about 15%, less than about 10%, or less than about 5%, based on a syringeability test in which a crushed solid oral dosage form is dissolved with about 10 ml of tap water with or without agitation at room temperature and the resultant solution is aspirated with a 18, 22, 25, or 27 gauge needle.

The invention also includes a solid oral dosage form comprising an opioid analgesic composition in an immediate release form and a gelling agent composition in a delayed release form,

wherein the solid oral dosage form is free of or substantially free of an opioid analgesic composition in an extended release form,

wherein the gelling agent composition comprises roller compacted and bottom sprayed granules, and

wherein the recovery of the opioid analgesic is less than about 30%, less than about 25%, or less than about 5%, based on a syringeability test in which a crushed solid oral dosage form is dissolved with about 10 ml of tap water with or without agitation at room temperature and the resultant solution is aspirated with a 18, 22, 25, or 27 gauge needle.

A solid oral dosage form of the invention, in a certain embodiment, comprises an active agent composition in an immediate release form and a gelling agent composition in delayed release form, wherein the solid oral dosage form is free of or substantially free of an active agent composition in an extended release form. The active agent can be a drug selected from the group consisting of opioid agonists, tranquilizers, CNS depressants, CNS stimulants, sedative hypnotics, and mixtures thereof.

The invention is also drawn to a method of treating a disease or condition through administering a solid oral dosage form of the invention. In certain embodiments, the solid oral dosage form is suitable for use in treating pain.

The invention is also drawn to a process for preparing a solid oral dosage form as above discussed, which comprises steps, such as (i) preparing a delayed release gelling agent composition; (ii) blending the delayed release gelling agent composition with an active agent composition; and (iii) compressing the blend into a tablet. In one embodiment, the preparing step comprises enteric coating a gelling agent, which can be performed by a method including such as, rotor powder layering, fluid bed granulation, roller compaction, fluid bed coating, and combination thereof.

The solid oral dosage form of the invention can also be prepared by a process comprising (i) preparing one or more particles; (ii) coating the one or more particles with an opioid analgesic composition; and (iii) blending the one or more particles coated with opioid analgesic composition with a delayed release gelling agent composition, wherein the solid oral dosage form comprises an opioid analgesic composition in an immediate release form, and wherein the solid oral dosage form is free of or substantially free of an opioid analgesic composition in an extended release form. One instance provides that the process further comprises a step of (iv) compressing the blend into a tablet.

The solid oral dosage form of the invention can also be prepared by a process comprising (i) preparing one or more delayed release gelling agent composition particles; and (ii) coating the one or more delayed release gelling agent particles with an opioid analgesic composition, wherein the solid oral dosage form comprises an opioid analgesic in an immediate release form, and wherein the solid oral dosage form is free of or substantially free of an opioid analgesic composition in an extended release form. One instance provides that the process further comprises a step of (iii) compressing the one or more coated particles into a tablet.

In addition, the invention provides a process of preparing a solid oral dosage form above discussed, which comprises steps such as: (i) preparing one or more delayed release gelling agent particles; and (ii) dispersing an opioid analgesic composition and the one or more delayed release gelling agent particles in a matrix, wherein the solid oral dosage form comprises an opioid analgesic composition in an immediate release form, and wherein the solid oral dosage form is free of or substantially free of an opioid analgesic composition in an extended release form.

### DEFINITIONS

As used herein, the singular forms “a,” “an,” and “the” include plural references unless the context clearly indicates otherwise. Thus, for example, reference to “an active agent susceptible to abuse” includes a single active agent as well as a mixture of two or more different active agents, and reference to a “gelling agent” includes a single gelling agent as well as a mixture of two or more different gelling agents, and the like.

As used herein, the term “about” in connection with a measured quantity, refers to the normal variations in that measured quantity, as expected by one of ordinary skill in the art in making the measurement and exercising a level of care commensurate with the objective of measurement and the precision of the measuring equipment. In certain embodiments, the term “about” includes the recited number ±10%, such that “about 10” would include from 9 to 11.

The terms “abuse-deterrent” and “tamper-resistant” refer to dosage forms that provide at least some physical and/or chemical barriers such as deterrence or resistance to, for example, crushing, chewing, cutting, grating or grinding of the dosage form, or extraction of the opioid from the dosage form using common solvents (e.g., water, simulated biological media, alcohol or organic solvents), or any combination thereof. The dosage forms may include agonist/antagonist combinations to interfere with, reduce or defeat the euphoria associated with abuse. The dosage forms may deter or be resistant to abuse even if they do not fully prevent abuse.

The term “recovery” means the amount of drug obtained from the resultant solution of a tampered dosage form (e.g., crushing and mixing in 5 mL solvent) upon aspiration with a needle, e.g., a 27 gauge needle.

As used herein, the terms “active agent,” “active ingredient,” “pharmaceutical agent,” and “drug” refer to any material that is intended to produce a therapeutic, prophylactic, or other intended effect, whether or not approved by a government agency for that purpose. These terms with respect to specific agents include all pharmaceutically active agents, all pharmaceutically acceptable salts thereof, complexes, stereoisomers, crystalline forms, co-crystals, ether, esters, hydrates, solvates, and mixtures thereof, where the form is pharmaceutically active.

As used herein, the terms “therapeutically effective” refers to the amount of drug or the rate of drug administration needed to produce a desired therapeutic result.

As used herein, the terms “prophylactically effective” refers to the amount of drug or the rate of drug administration needed to produce a desired prophylactic result.

As used herein, the term “antitussive amount” refers to an amount of a drug sufficient to relieve, suppress, or reduce the frequency of coughing.

As used herein, the term “analgesically effective amount” refers to an amount of a drug sufficient to provide analgesia.

As used herein, the term “stereoisomers” is a general term for all isomers of individual molecules that differ only in the orientation of their atoms in space. It includes enantiomers and isomers of compounds with one or more chiral centers that are not mirror images of one another (diastereomers).

The term “enantiomer” or “enantiomeric” refers to a molecule that is non-superimposable on its mirror image and hence optically active wherein the enantiomer rotates the plane of polarized light in one direction by a certain degree, and its mirror image rotates the plane of polarized light by the same degree but in the opposite direction.

The term “chiral center” refers to a carbon atom to which four different groups are attached.

The term “racemic” refers to a mixture of enantiomers.

The term “resolution” refers to the separation or concentration or depletion of one of the two enantiomeric forms of a molecule.

The term “patient” refers to a subject, particularly a human, who has presented a clinical manifestation of a particular symptom or symptoms suggesting the need for treatment, who is treated preventatively or prophylactically for a condition, or who has been diagnosed with a condition to be treated. The term “subject” is inclusive of the definition of the term “patient” and does not exclude individuals who are otherwise healthy.

“Pharmaceutically acceptable salts” include, but are not limited to, inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate and the like; amino acid salts such as arginate, asparaginate, glutamate and the like; metal salts such as sodium salt, potassium salt, cesium salt and the like; alkaline earth metals such as calcium salt, magnesium salt and the like; and organic amine salts such as triethylamine salt, pyridine salt, picoline salt, ethanolamine salt, triethanolamine salt, discyclohexylamine salt, N,N′-dibenzylethylenediamine salt and the like.

The term “ppm” as used herein means “parts per million”.

The term “compression” refers to a tableting process where the tablet or any other compressed dosage form is made by a process including blending the components of the formulation and compressing the blend to form the formulation.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

The term “Condition” or “Conditions” refers to those medical conditions that can be treated or prevented by administration to a subject of an effective amount of an active agent (such as an opioid analgesic or pharmaceutically acceptable salt thereof), such as acute and chronic pain, pulmonary edema, cough, diarrhea, inflammation or inflammatory disease, etc.

The terms “treatment of” and “treating” includes the lessening of the severity of or cessation of a Condition, e.g., pain. In one embodiment, “treating” or “treatment of” may include inhibiting, for example decreasing, the overall frequency of episodes of pain.

The terms “prevention of” and “preventing” includes the avoidance of the onset of a Condition, e.g., pain.

The term “free or substantially free of an extended release opioid analgesic composition” or “free or substantially free of an extended release active agent composition” or “free or substantially free of an opioid analgesic composition in an extended release form” or “free or substantially free of an active agent composition in an extended release form” refers to solid oral dosage forms in which about 15% or less, about 10% or less, about 5% or less, of the opioid analgesic or active agent in the solid oral dosage form is released in accordance with an extended release profile (where an extended release profile may refer, e.g., to the amount of active agent released at a time period of greater than 4 hours as measured by in-vitro dissolution in a USP Apparatus 1 (#40 mesh basket) in 900 ml 0.1N HCl at room temperature).

The term “extended release” refers to an active agent that is released over a period of time, e.g., to provide a once daily or twice daily dosage form. The terms “extended release active agent composition,” “active agent composition in an extended release form,” “extended release active agent,” and “active agent in an extended release form” are used interchangeably.

The terms “delayed release gelling agent composition” and “gelling agent composition in a delayed release form” are used interchangeably. These terms refer to the gelling agent being released after the occurrence of an event. The event may be the passage of time, a trigger such as change in pH, or any other comparable event as understood by one of ordinary skill in the art.

The term “release” as used with respect to the gelling agent refers to at least a partial release of the gelling agent that causes an increase in viscosity.

The term “immediate release” refers to the release of at least 85%, at least 90%, or at least 95% of an active agent in a time from of 5 minutes, 15 minutes, 30 minutes, 45 minutes or 60 minutes, as measured by in-vitro dissolution in a USP Apparatus 1 (#40 mesh basket) in 900 ml 0.1N HCl at room temperature.

The terms “immediate release active agent composition” and “active agent composition in an immediate release form” are used interchangeably.

The terms “immediate release opioid analgesic composition” and “opioid analgesic composition in an immediate release form” are used interchangeably.

Viscosity measurements disclosed herein can be measured using a rotational viscometer (e.g. a Brookfield RV viscometer available from Brookfield Engineering, Middleboro, Mass. USA or equivalent) with the spindle number that corresponds to the viscosity range to be measured (e.g., spindle numbers 1-7 for viscosities ranging between 100-160,000 cP). Viscosity measurements can be measured in accordance with the United States Pharmacopeia (USP) monograph for Carbomer Homopolymer (USP40-NF35) as recited in the Aug. 1, 2017 version (which is hereby incorporated by reference), or a comparable method as understood by one with ordinary skill in the art.

Syringeability tests as used herein refer to tablets that are tested for syringeability of liquid post dissolution using 5 mL and 10 mL tap water at room temperature. Tablets were tested in an intact and in a crushed form. The aspiration is performed over a duration of about 1-60 minutes in an iterative process with a 18, 22, 25, or 27 gauge needle. Aspirated volumes of water are noted and assayed for content of active agent aspirated.

Disintegration tests refer to tablets that are tested for active agent released in 0.1 N HCl and in water in accordance with the United States Pharmacopeia (USP) disintegration test procedure described in the official version of Apr. 1, 2006 (which is hereby incorporated by reference).

For purposes of the present invention, the “in-vitro dissolution test in a USP Apparatus 1 (#40 mesh basket)” is used in a slightly modified form, by equipping the USP Apparatus 1 basket with a retaining spring placed in the upper part of the basket (above the dosage form), to reduce the propensity of the tested dosage forms, once hydrated in the dissolution medium, to stick to the solid underside of the top of the basket or the base of the shaft. For example, a passivized stainless steel 316 spring, 1.5-cm outside diameter and 2-cm length can be used.

### Dosage Forms

According to various embodiments, the present disclosure is related to immediate release solid oral dosage forms comprising an immediate release opioid analgesic composition and a delayed release gelling agent composition. In certain embodiments, the delayed release gelling agent composition does not interfere with the immediate release characteristics of the active agent when the dosage form is orally administered intact as directed. In certain embodiments, the delayed release gelling agent composition, the opioid analgesic, or both may be in a form of one or more particles. In certain embodiments, the delayed release gelling agent composition may be in a form of one or more particles dispersed in a matrix material comprising an opioid analgesic. In other embodiments, the opioid analgesic composition may be in a form of one or more particles dispersed in a matrix material comprising the delayed release gelling agent composition. In certain embodiments, the delayed release gelling agent composition may be in the form of one or more particles and the opioid analgesic composition may be in the form of one or more particles such that the various kinds of particles are dispersed in a matrix material. In other embodiments, the delayed release gelling agent composition may be in a form of one or more particles and the opioid analgesic composition may be coated onto the one or more delayed release gelling agent particles.

In certain embodiments, wherein the active agent composition is in the form of one or more particles, the active agent (e.g., opioid analgesic) composition may be coated over an inert particle or bead core to form an active agent layer on each particle in the dosage form. In certain embodiments, instead of an inert particle or bead core, the core of the particles may be formed from a delayed release gelling agent composition, an aversive agent, an excipient or any combination thereof. Active agents may be present in any one or more of the layers of the multiparticulates.

In certain embodiments, the one or more opioid analgesic particles and the one or more delayed release gelling agent particles may be compressed into a tablet, or may be contained in a pharmaceutically acceptable capsule.

One or more aversive agents may also be included in the dosage form. For example, the aversive agent may be included in one or more particles, which may be combined in a dosage form including the delayed release gelling agent composition and active agent composition. In other embodiments, the one or more aversive agents may be included in the delayed release gelling agent composition, or with the active agent composition, or with both compositions.

According to certain embodiments, the immediate release solid oral dosage forms may release the active agent in accordance with the immediate release dissolution profile when the dosage form is taken as intended. However, when the dosage form is tampered with, for instance by mixing the dosage form in from about 0.5 ml to about 10 ml of water (or any other aqueous solution having a pH greater than 5.5), the delayed release gelling agent composition may be at least partially released to increase the viscosity of the tampered dosage form, making the active agent (e.g., opioid analgesic) composition unsuitable for parenteral (e.g., intravenous) administration.

The immediate release profile may be altered, for example, by varying the formulation of the dosage form, such as by altering the matrix material, by altering the types and amounts of excipients added, by altering the type and amount of aversive agents added, by the inclusion of additional ingredients, by altering the method of manufacture, etc. For instance, the immediate release profile may be influenced by the inclusion of release-modifying agents, which may function as pore-formers, may be organic or inorganic, and may include materials that can be dissolved, extracted or leached from the dosage form in the environment of use. The pore-formers may include one or more hydrophilic materials such as hydroxypropylmethylcellulose, lactose, and mixtures of any of the foregoing.

In certain embodiments, the dosage form (e.g., in the form of a matrix) may include a hydrophobic material, including, but is not limited to, digestible, long chain (C₈-C₅₀, especially C₁₂-C₄₀), substituted or unsubstituted hydrocarbons, such as natural or synthetic waxes (such as beeswax, glycowax, castor wax and carnauba wax), fatty alcohols (such as lauryl, myristyl, stearyl, cetyl or preferably cetostearyl alcohol), fatty acids, including, but not limited to, fatty acid esters, fatty acid glycerides (mono-, di-, and tri-glycerides), hydrogenated fats, hydrocarbons, normal waxes, stearic acid, stearyl alcohol and hydrophobic and hydrophilic materials having hydrocarbon backbones.

In addition to the above ingredients, the dosage form may also contain suitable quantities of other pharmaceutically acceptable excipients, e.g., diluents, lubricants, binders, granulating aids, and glidants that are known to those of ordinary skill in the art.

According to various embodiments, the immediate release solid oral dosage forms (e.g., compressed tablets) may have a hardness of about 2 Kp to about 20 Kp, or about 5 Kp to about 18 Kp.

In further embodiments, the immediate release solid oral dosage form may be in any suitable form for administration. The dosage form may be in the form of compressed tablets, gelcaps, capsules, caplets, granules, lozenges, bulk powders, film, or extrudate. A tablet may have any suitable shape including, but not limited to, a round shape, caplet shape, or a troche shape.

For instance, the entire dosage form, including any additional ingredients besides the active agent composition and delayed release gelling agent composition, may be in the form of a unitary dosage form such as a tablet. The tablet may be prepared by compression to form a compressed tablet. The tablet core may include the active agent composition dispersed in one or more excipients and/or aversive agents. The tablet core may also be formed from multiparticulates as described above that are compressed into a tablet shape.

In other embodiments, the entire dosage form, including any additional ingredients besides the active agent composition and delayed release gelling agent composition, may be in the form of a plurality of particles. The plurality of particles may be contained in a pharmaceutically acceptable capsule, such as a gelatin capsule.

When the dosage forms are in the form of a tablet, such tablets may be compressed, tablet triturates, sugar-coated, film-coated, multiply compressed, or multi-layered.

When the dosage forms are in a multiparticulate formulation, the unit dose of a multiparticulate dosage form of the present invention may include without limitation, from about 2 to about 75 particles; from about 10 to about 50 particles; from about 15 to about 25 particles; or from about 10 to about 50 particles. In other embodiments, a unit dose of an immediate release dosage form of the present invention may include without limitation, from about 50 to about 500 particles; from about 75 to about 350 particles; from about 100 to about 300 particles; or from about 150 to about 250 particles.

According to various embodiments, the solid oral dosage form may include a therapeutically effective amount, an antitussive amount, or an analgesically effective amount of the active agent (e.g., opioid analgesic).

### Release Rates

The solid oral dosage forms disclosed herein can provide an immediate release of the active agent. In certain embodiments, the solid oral dosage forms disclosed herein provide an immediate release of an opioid analgesic and are free of or substantially free of an extended release of the opioid analgesic.

In certain embodiments, the solid oral dosage forms disclosed herein release at least about 80%, at least about 85%, at least about 90%, at least about 95%, or at least about 98% of the opioid analgesic within 15 minutes as measured by in-vitro dissolution in a USP Apparatus 1 (#40 mesh basket) in 900 ml 0.1N HCl at about 37° C.

In certain embodiments, the solid oral dosage forms disclosed herein release at least about 80%, at least about 85%, at least about 90%, at least about 95%, or at least about 98% of the opioid analgesic within 30 minutes as measured by in-vitro dissolution in a USP Apparatus 1 (#40 mesh basket) in 900 ml 0.1N HCl at 37° C.

In certain embodiments, the solid oral dosage forms disclosed herein release at least about 80%, at least about 85%, at least about 90%, at least about 95%, or at least about 98% of the opioid analgesic within 45 minutes as measured by in-vitro dissolution in a USP Apparatus 1 (#40 mesh basket) in 900 ml 0.1N HCl at 37° C.

In certain embodiments, the solid oral dosage forms disclosed herein release at least about 80%, at least about 85%, at least about 90%, at least about 95%, or at least about 98% of the opioid analgesic within 60 minutes as measured by in-vitro dissolution in a USP Apparatus 1 (#40 mesh basket) in 900 ml 0.1N HCl at room temperature.

In some embodiments, the solid oral dosage forms disclosed herein comprise a gelling agent composition in a delayed release form, wherein the gelling agent composition comprises a gelling agent and an enteric coating. The gelling agent composition may be prepared by a method selected from the group consisting of rotor powder layering, fluid bed granulation, roller compaction, fluid bed coating (wurster coating), and combination thereof.

In certain embodiments, the immediate release solid oral dosage form disclosed herein comprises an immediate release opioid analgesic composition and a delayed release gelling agent composition, wherein the solid oral dosage form is free of or substantially free of an extended release opioid analgesic composition, wherein the delayed release gelling agent composition comprises rotor layered and enteric coated pellets comprising about 10% (w/w) enteric material based on total weight of the pellets, and wherein the recovery of the opioid analgesic is less than about 15% based on a syringeability test whereby an intact solid oral dosage form is dissolved with about 5 ml to about 10 ml of tap water without agitation at room temperature and the resultant solution is aspirated with a 18, 22, 25, or 27 gauge needle.

In certain embodiments, the immediate release solid oral dosage form disclosed herein comprises an immediate release opioid analgesic composition and a delayed release gelling agent composition, wherein the solid oral dosage form is free of or substantially free of an extended release opioid analgesic composition, wherein the delayed release gelling agent composition comprises rotor layered and enteric coated pellets comprising about 10% (w/w) enteric material based on total weight of the pellets, and wherein the recovery of the opioid analgesic is less than about 5% based on a syringeability test whereby an intact solid oral dosage form is dissolved with about 5 ml to about 10 ml of tap water with agitation at room temperature and the resultant solution is aspirated with a 18, 22, 25, or 27 gauge needle.

In certain embodiments, the immediate release solid oral dosage form disclosed herein comprises an immediate release opioid analgesic composition and a delayed release gelling agent composition, wherein the solid oral dosage form is free of or substantially free of an extended release opioid analgesic composition, wherein the delayed release gelling agent composition comprises rotor layered and enteric coated pellets comprising about 30% (w/w) enteric material based on total weight of the pellets, and wherein the recovery of the opioid analgesic is less than about 20% based on a syringeability test whereby an intact solid oral dosage form is dissolved with about 10 ml of tap water with or without agitation at room temperature and the resultant solution is aspirated with a 18, 22, 25, or 27 gauge needle.

In certain embodiments, the immediate release solid oral dosage form disclosed herein comprises an immediate release opioid analgesic composition and a delayed release gelling agent composition, wherein the solid oral dosage form is free of or substantially free of an extended release opioid analgesic composition, wherein the delayed release gelling agent composition comprises rotor layered and enteric coated pellets comprising about 30% (w/w) enteric material based on total weight of the pellets, and wherein the recovery of the opioid analgesic is less than about 10% based on a syringeability test whereby an intact solid oral dosage form is dissolved with about 5 ml of tap water without agitation at room temperature and the resultant solution is aspirated with a 18, 22, 25, or 27 gauge needle.

In certain embodiments, the immediate release solid oral dosage form disclosed herein comprises an immediate release opioid analgesic composition and a delayed release gelling agent composition, wherein the solid oral dosage form is free of or substantially free of an extended release opioid analgesic composition, wherein the delayed release gelling agent composition comprises rotor layered and enteric coated pellets comprising about 30% (w/w) enteric material based on total weight of the pellets, and wherein the recovery of the opioid analgesic is less than about 5% based on a syringeability test whereby an intact solid oral dosage form is dissolved with about 5 ml of tap water with agitation at room temperature and the resultant solution is aspirated with a 18, 22, 25, or 27 gauge needle.

In certain embodiments, the immediate release solid oral dosage form disclosed herein comprises an immediate release opioid analgesic composition and a delayed release gelling agent composition, wherein the solid oral dosage form is free of or substantially free of an extended release opioid analgesic composition, wherein the delayed release gelling agent composition comprises top sprayed granules, and wherein the recovery of the opioid analgesic is less than about 2% based on a syringeability test whereby an intact solid oral dosage form is dissolved with about 5 ml to about 10 ml of tap water with or without agitation at room temperature and the resultant solution is aspirated with a 18, 22, 25, or 27 gauge needle.

In certain embodiments, the immediate release solid oral dosage form disclosed herein comprises an immediate release opioid analgesic composition and a delayed release gelling agent composition, wherein the solid oral dosage form is free of or substantially free of an extended release opioid analgesic composition, wherein the delayed release gelling agent composition comprises roller compacted and bottom sprayed (enteric coated) granules, wherein the recovery of the opioid analgesic is less than about 40%, less than about 30%, less than about 10%, or less than about 8% based on a syringeability test whereby an intact solid oral dosage form is dissolved with about 5 ml to about 10 ml of tap water with or without agitation at room temperature and the resultant solution is aspirated with a 18, 22, 25, or 27 gauge needle.

In certain embodiments, the immediate release solid oral dosage form disclosed herein comprises an immediate release opioid analgesic composition and a delayed release gelling agent composition, wherein the solid oral dosage form is free of or substantially free of an extended release opioid analgesic composition, wherein the delayed release gelling agent composition comprises rotor granulated and enteric coated pellets comprising about 30% (w/w) enteric material based on total weight of the pellets, and wherein the recovery of the opioid analgesic is less than about 15%, less than about 10%, or less than about 5% based on a syringeability test whereby a crushed solid oral dosage form is dissolved with about 10 ml of tap water with or without agitation at room temperature and the resultant solution is aspirated with a 18, 22, 25, or 27 gauge needle.

In certain embodiments, the immediate release solid oral dosage form disclosed herein comprises an immediate release opioid analgesic composition and a delayed release gelling agent particles, wherein the solid oral dosage form is free of or substantially free of an extended release opioid analgesic composition, wherein the delayed release gelling agent composition comprises roller compacted and bottom sprayed (enteric coated) granules, and wherein the recovery of the opioid analgesic is less than about 30%, less than about 25%, or less than about 5% based on a syringeability test whereby a crushed solid oral dosage form is dissolved with about 10 ml of tap water with or without agitation at room temperature and the resultant solution is aspirated with a 18, 22, 25, or 27 gauge needle.

### Active Agents

The immediate release solid oral dosage forms according to the disclosure include various active agents and their pharmaceutically acceptable salts thereof. Pharmaceutically acceptable salts include, but are not limited to, inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate, and the like; amino acid salts such as arginate, asparginate, glutamate and the like, and metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like.

According to certain embodiments, any of the following active agents can be used in the immediate release solid oral dosage forms disclosed herein: ACE inhibitors, adenohypophoseal hormones, adrenergic neuron blocking agents, adrenocortical steroids, inhibitors of the biosynthesis of adrenocortical steroids, alpha-adrenergic agonists, alpha-adrenergic antagonists, selective alpha-two-adrenergic agonists, analgesics, anti-pyretics, anti-inflammatory agents, androgens, local and general anesthetics, anti-addictive agents, anti-androgens, anti-arrhythmic agents, anti-cholinergic agents, anti-cholinesterase agents, anti-coagulants, anti-diuretic, anti-emetic agents, pro-kinetic agents, anti-estrogens, anti-fungal agents, anti-microbial agents, anti-migraine agents, anti-muscarinic agents, anti-neoplastic agents, anti-parasitic agents, anti-parkinson's agents, anti-platelet agents, anti-progestins, anti-schizophrenia agents, anti-thyroid agents, anti-viral agents, atypical anti-depressants, azaspirodecanediones, barbiturates, benzodiazepines, benzothiadiazides, beta-adrenergic agonists, beta-adrenergic antagonists, selective beta-one-adrenergic antagonists, selective beta-two-adrenergic agonists, bile salts, agents affecting volume and composition of body fluids, butyrophenones, agents affecting calcification, calcium channel blockers, cardiovascular drugs, catecholamines and sympathomimetic drugs, cholinergic agonists, cholinesterase reactivators, contraceptive agents, dermatological agents, diphenylbutylpiperidines, ergot alkaloids, estrogens, ganglionic blocking agents, ganglionic stimulating agents, hydantoins, agents for control of gastric acidity and treatment of peptic ulcers, hematopoietic agents, hormones, 5-hydroxytryptamine antagonists, drugs for the treatment of hyperlipoproteinemia, hypnotics, immunosupressive agents, methylxanthines, moncamine oxidase inhibitors, neuromuscular blocking agents, organic nitrates, opioid agonists, opioid antagonists, pancreatic enzymes, phenothiazines, progestins, prostaglandins, agents for the treatment of psychiatric disorders, retinoids, sodium channel blockers, agents for spasticity and acute muscle spasms, succinimides, testosterones, thioxanthines, thrombolytic agents, thyroid agents, tricyclic antidepressants, inhibitors of tubular transport of organic compounds, drugs affecting uterine motility, and mixtures thereof.

In certain embodiments, the active agent for the immediate release solid oral dosage forms disclosed herein is an active agent susceptible to abuse. In certain embodiments, the active agent may be selected from the group consisting of opioid agonists, tranquilizers, CNS depressants, CNS stimulants, sedative hypnotics, and mixtures thereof.

According to certain embodiments, active agents may include an opioid analgesic. Useful opioid analgesics for the immediate release solid oral dosage forms disclosed herein include, but are not limited to, alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, dihydroetorphine, fentanyl and derivatives, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanil, tilidine, tramadol, and the pharmaceutically acceptable salts, hydrates, solvates and prodrugs thereof, mixtures of any of the foregoing, and the like.

In certain embodiments, the opioid analgesic for the immediate release solid oral dosage forms disclosed herein may be selected from the group consisting of morphine, hydromorphone, hydrocodone, oxycodone, codeine, levorphanol, meperidine, dihydrocodeine, dihydromorphine, oxymorphone, fentanyl, buprenorphine pharmaceutically acceptable salts thereof, solvates thereof, prodrugs thereof, and mixtures thereof.

Examples of other possible active agents include, but are not limited to, antihistamines (e.g., dimenhydrinate, diphenhydramine, chlorpheniramine and dexchlorpheniramine maleate), non-steroidal anti-inflammatory agents (e.g., aspirin, celecoxib, Cox-2 inhibitors, ibuprofen, indomethacin, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid, tolfenamic acid, diflurisal, flufenisal, piroxicam, sudoxicam, isoxicam, pharmaceutically acceptable salts thereof and mixtures thereof) and acetaminophen, anti-emetics (e.g., metoclopramide, methylnaltrexone), anti-epileptics (e.g., phenyloin, meprobmate and nitrazepam), vasodilators (e.g., nifedipine, papaverine, diltiazem and nicardipine), anti-tussive agents and expectorants (e.g. codeine phosphate), anti-asthmatics (e.g. theophylline), antacids, anti-spasmodics (e.g. atropine, scopolamine), antidiabetics (e.g., insulin), diuretics (e.g., ethacrynic acid, bendrofluthiazide), anti-hypotensives (e.g., propranolol, clonidine), antihypertensives (e.g., clonidine, methyldopa), bronchodilatiors (e.g., albuterol), steroids (e.g., hydrocortisone, triamcinolone, prednisone), antibiotics (e.g., tetracycline), antihemorrhoidals, hypnotics, psychotropics, antidiarrheals, mucolytics, sedatives, decongestants (e.g. pseudoephedrine), laxatives, vitamins, stimulants (including appetite suppressants such as phenylpropanolamine) and cannabinoids, as well as pharmaceutically acceptable salts, hydrates, solvates, and prodrugs thereof.

The active agent that may also be a benzodiazepine, barbiturate, stimulants, or mixtures thereof. The term “benzodiazepines” refers to a benzodiazepine and drugs that are derivatives of a benzodiazepine that are able to depress the central nervous system. Benzodiazepines include, but are not limited to, alprazolam, bromazepam, chlordiazepoxide, clorazepate, diazepam, estazolam, flurazepam, halazepam, ketazolam, lorazepam, nitrazepam, oxazepam, prazepam, quazepam, temazepam, triazolam, methylphenidate as well as pharmaceutically acceptable salts, hydrates, solvates, prodrugs and mixtures thereof. Benzodiazepine antagonists that can be used as active agent include, but are not limited to, flumazenil as well as pharmaceutically acceptable salts, hydrates, solvates and mixtures thereof.

The term “barbiturates” refers to sedative-hypnotic drugs derived from barbituric acid (2,4,6,-trioxohexahydropyrimidine). Barbiturates include, but are not limited to, amobarbital, aprobarbotal, butabarbital, butalbital, methohexital, mephobarbital, metharbital, pentobarbital, phenobarbital, secobarbital as well as pharmaceutically acceptable salts, hydrates, solvates, prodrugs, and mixtures thereof. Barbiturate antagonists that can be used as active agent include, but are not limited to, amphetamines as well as pharmaceutically acceptable salts, hydrates, solvates and mixtures thereof.

The term “stimulants” includes, but is not limited to, amphetamines such as dextroamphetamine resin complex, dextroamphetamine, methamphetamine, methylphenidate, as well as pharmaceutically acceptable salts, hydrates, and solvates and mixtures thereof. Stimulant antagonists that can be used as active agent include, but are not limited to, benzodiazepines, as well as pharmaceutically acceptable salts, hydrates, solvates and mixtures thereof.

In certain embodiments, the immediate release solid oral dosage forms disclosed herein may comprise from about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, or about 7% to about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 60%, about 70%, or about 80% (w/w) active agent (e.g., opioid analgesic) per dosage form. In certain embodiments, the immediate release solid oral dosage forms disclosed herein may comprise from about 0.1% to about 80%, from about 0.5% to about 30%, or from about 1% to about 10% (w/w) of an active agent (e.g., opioid analgesic) per dosage form.

### Delayed Release Gelling Agent Composition

In certain embodiments, the delayed release gelling agent composition disclosed herein may comprise a gelling agent and an enteric material. In certain embodiments, the delayed release gelling agent composition may be in a form of one or more particles. Each delayed release gelling agent particle may comprise (i) the gelling agent coated with an enteric material, (ii) an inert core (e.g., bead) coated with the gelling agent and overcoated with an enteric material, or (iii) the gelling agent dispersed in an enteric matrix material. In certain embodiments, in addition to the delayed release gelling agent composition, the dosage form may include an amount of gelling agent in immediate release form that does not interfere with the immediate release characteristics of the dosage form.

In certain embodiments, the gelling agent used in the immediate release solid oral dosage forms disclosed herein may include, but not be limited to, selected from, starch and starch derivatives, cellulose derivatives (e.g., sodium carboxymethyl cellulose, methylcellulose, hydroxyethylcellulose, hydroxypropylcellulose, hydroxypropylmethylcellulose), attapulgites, bentonites, dextrins, alginates, carrageenan, gums (e.g., gum tragacanth, gum acacia, guar gum, and xanthan gum), pectin, gelatin, kaolin, high molecular weight polymer of acrylic acid cross-linked with allyl ethers of polyalcohols such as carbomers (also referred to as crosslinked polyacrylic acid), polyvinylpyrrolidone, polyethylene oxide, polyvinyl alcohol, curdlan, furcelleran, egg white powder, lacto albumin, soy protein, chitosan, surfactants, mixed surfactant/wetting agent systems, emulsifying agents, other polymeric materials, and mixtures thereof.

In certain embodiments, the gelling agent may be selected from the group consisting of polyethylene oxide, xanthan gum, carbomers, polysaccharides, and mixtures thereof.

In certain embodiments, the gelling agent may be a polymer (e.g., a gelling polymer), such as a polysaccharide, specifically a microbial polysaccharide such as xanthan gum. In certain embodiments, the gelling agent may be an anionic polymer in a neutral pH aqueous solution such as a polyacrylic acid, specifically a carbomer homopolymer. In certain embodiments, the gelling agent may be xanthan gum and a carbomer homopolymer.

The gelling agent may be included in the immediate release solid oral dosage forms in an amount such that the viscosity of the dosage form mixed (crushed or intact) with from about 0.5 ml to about 10 ml of tap water at room temperature prevents or reduces the ability of the active agent (e.g., opioid analgesic) from being drawn up into a syringe, or from being systemically absorbed when administered by parenteral or nasal route. In certain embodiments, the viscosity of the tampered dosage form may make the dosage form unsuitable for parenteral or intravenous administration.

In some embodiments, the viscosity (e.g., measured from a rotational viscometer) of a solution obtained from an intact solid oral dosage form at 5 minutes in about 0.5 ml to about 10 ml water at room temperature is about 10 cP or more, about 25 cP or more, about 50 cP or more, about 75 cP or more, about 100 cP or more, about 125 cP or more to about 150 cP or more, about 175 cP or more, about 200 cP or more, about 300 cP or more, about 400 cP or more, about 500 cP or more, about 750 cP or more, about 1000 cP or more, about 2000 cP or more, about 3000 cP or more, about 4000 cP or more, about 5000 cP or more, about 7500 cP or more, about 10,000 cP or more, about 15,000 cP or more, about 20,000 cP or more, about 25,000 cP or more, about 50,000 cP or more, about 75,000 cP or more, about 100,000 cP or more, about 125,000 cP or more, or about 150,000 cP or more.

In some embodiments, the viscosity (e.g., measured from a rotational viscometer) of a solution obtained from an intact solid oral dosage form at 5 minutes in about 0.5 ml to about 10 ml water at room temperature may be in the range of about 10-150,000 cP, about 25-10,000 cP, about 25-1000 cP, about 50-1000 cP, about 75-1000 cP, about 25-500 cP, about 50-500 cP, about 75-500 cP, about 25-200 cP, about 50-200 cP, or about 75-200 cP.

In some embodiments, the viscosity of a solution obtained from a tampered solid oral dosage form at 2 minutes in about 0.5 ml to about 10 ml water at room temperature is about 10 cP or more, about 25 cP or more, about 50 cP or more, about 75 cP or more, about 100 cP or more, or about 125 cP or more about 150 cP or more, about 175 cP or more, about 200 cP or more, about 300 cP or more, about 400 cP or more, about 500 cP or more, about 750 cP or more, about 1000 cP or more, about 2000 cP or more, about 3000 cP or more, about 4000 cP or more, about 5000 cP or more, about 7500 cP or more, about 10,000 cP or more, about 15,000 cP or more, about 20,000 cP or more, about 25,000 cP or more, about 50,000 cP or more, about 75,000 cP or more, about 100,000 cP or more, about 125,000 cP or more, or about 150,000 cP or more. In some embodiments, the viscosity of a solution obtained from a tampered solid oral dosage form at 2 minutes in about 0.5 ml to about 10 ml water at room temperature may be in the range of about 10-150,000 cP, about 25-10,000 cP, about 25-1000 cP, about 50-1000 cP, about 75-1000 cP, about 25-500 cP, about 50-500 cP, about 75-500 cP, about 25-200 cP, about 50-200 cP, or about 75-200 cP.

In some embodiments, the viscosity obtained from an intact solid oral dosage form at 5 minutes in about 0.5 ml to about 10 ml 0.1N HCl at room temperature is about 50 cP or less, about 40 cP or less, about 30 cP or less, about 20 cP or less, about 10 cP or less, about 5 cP or less, or about 2 cP or less.

In some embodiments, the viscosity obtained from a tampered solid oral dosage form at 5 minutes in about 0.5 ml to about 10 ml 0.1N HCl at room temperature is 50 cP or less, about 40 cP or less, about 30 cP or less, about 20 cP or less, about 10 cP or less, about 5 cP or less, or about 2 cP or less.

In some embodiments, the viscosity of a solution obtained from an intact solid oral dosage form at 5 minutes in about 0.5 ml to about 10 ml of 0.1N HCl at room temperature is within about 30%, about 20%, about 10%, or about 5% of the viscosity of a solution obtained from a tampered solid oral dosage form at 5 minutes in about 0.5 ml to about 10 ml of 0.1N HCl at room temperature.

In certain embodiments, the weight amount of gelling agent contained in the immediate release dosage form of the present invention is not more than the weight amount of active agent (e.g., opioid analgesic). In other embodiments, the weight amount of gelling agent contained in the immediate release dosage forms of the present invention is less than the weight amount of active agent. In further embodiments, the weight amount of gelling agent contained in the immediate release dosage forms of the present invention is more than the weight amount of active agent.

In certain embodiments, the immediate release dosage forms of the present invention contain a weight ratio of active agent (e.g., opioid analgesic) to gelling agent from about 1:40 to about 40:1; from about 1:35 to about 35:1; from about 1:30 to about 30:1; from about 1:25 to about 25:1; from about 1:20 to about 20:1; from about 1:15 to about 15:1; from about 1:10 to about 10:1; from about 1:8 to about 8:1; from about 1:5 to about 5:1; from about 1:3 to about 3:1; from about 1:1.5 to about 1.5:1; from about 1:1.25 to about 1.25:1; 1:1 to about 40:1; from about 1:1 to about 35:1; from about 1:1 to about 30:1; from about 1:1 to about 25:1; from about 1:1 to about 20:1; from about 1:1 to about 15:1; from about 1:1 to about 10:1; from about 1:1 to about 8:1; from about 1:1 to about 5:1; from about 1:1 to about 3:1; from about 1:1 to about 1.5:1; from about 1:1 to about 1.25:1; 1:40 to about 1:1; from about 1:35 to about 1:1; from about 1:30 to about 1:1; from about 1:25 to about 1:1; from about 1:20 to about 1:1; from about 1:15 to about 1:1; from about 1:10 to about 1:1; from about 1:8 to about 1:1; from about 1:5 to about 1:1; from about 1:3 to about 1:1; from about 1:1.5 to about 1:1; or from about 1:1.25 to about 1:1.

In certain embodiments, the immediate release solid oral dosage forms disclosed herein may comprise from about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, or about 7% to about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, or about 60% (w/w) gelling agent based on the weight of the dosage form. In certain embodiments, the dosage form may contain from about 0.1% to about 60%, from about 0.5% to about 20%, or from about 1% to about 10% gelling agent based on the total weight of the dosage form.

According to some embodiments, the delayed release gelling composition may comprise one or more of rotor layered and enteric coated pellets, top sprayed granules, roller compacted pellets, bottom sprayed granules (e.g., wurster coated granules), or combinations thereof.

In certain embodiments, when the delayed release gelling agent composition comprises top spray granules, about 90% to about 100%, about 95% to about 100%, or about 99% to about 100% of the top spray granules incorporated into the immediate release solid oral dosage form may be retained in one or more of a 100 mesh screen, a 200 mesh screen, and a pan. In certain embodiments, when the delayed release gelling agent composition comprises top spray granules, about 20% to about 50%, about 30% to about 45%, or about 40% of the top sprat granules incorporated into the immediate release solid oral dosage form may be retained in a 100 mesh screen. In certain embodiments, when the delayed release gelling agent composition comprises top spray granules, about 20% to about 50%, about 30% to about 45%, or about 40% of the top sprat granules incorporated into the immediate release solid oral dosage form may be retained in a 200 mesh screen. In certain embodiments, when the delayed release gelling agent composition comprises top spray granules, about 10% to about 30%, about 15% to about 25%, or about 20% of the top spray granules incorporated into the immediate release solid oral dosage form may pass through a 200 mesh screen.

In certain embodiments, when the delayed release gelling agent composition comprises roller compacted pellets, the pellets may have a bulk density ranging from about 0.4 g/ml to about 0.6 g/ml, or a tapped density ranging from about 0.55 g/ml to about 0.65 g/ml. The term “tapped density” as used herein, refers to the increased bulk density attained after mechanically tapping the container containing the powder sample.

### Enteric Materials

In certain embodiments, the enteric material may overcoat the gelling agent(s) in one or more delayed release gelling agent particles. In other embodiments, the enteric material may be part of a matrix material (unitary or multiparticulate) in which the delayed release gelling agent composition may be dispersed.

In certain embodiments, the enteric material may (i) dissolve above a pH of about 5.5, (ii) not dissolve below a pH of about 5.5, or (iii) dissolve at a pH of 5.5 or higher and not dissolve below a pH of 5.5.

In certain embodiments, the enteric material used to coat the gelling agent may be selected from the group consisting of a cellulosic material, an acrylic polymer, a methacrylic polymer and mixtures thereof.

According to certain embodiments, the enteric material may be selected from the group consisting of methacrylic acid/methyl methacrylate, methacrylic acid/ethyl acrylate copolymers, methacrylic acid/methyl acrylate/methyl methacrylate copolymers, shellac, hydroxypropyl methylcellulose phthalate, hydroxyl propyl methyl cellulose acetate succinate, hydroxypropyl methyl cellulose trimellitate, cellulose acetate phthalates, polyvinyl acetate phthalates and mixtures thereof. In certain embodiments, the enteric material may be a methacrylic acid/ethyl acrylate copolymer.

According to certain embodiments, the dosage form may include from about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or about 10% to about 11%, about 12%, about 13%, about 14%, about 15%, about 16%, about 17%, about 18%, about 19%, about 20%, about 25%, about 30%, about 35%, about 40%, about 45%, or about 50% (w/w) enteric material based on the weight of the dosage form. In certain embodiments, the dosage form may contain from about 0.1% to about 50%, from about 1% to about 20%, or from about 2% to about 15% (w/w) enteric material based on the total weight of the dosage form.

According to certain embodiments where the solid oral dosage form comprises delayed release gelling agent composition in the form of one or more particles, each delayed release gelling agent particle may comprise from about 1%, about 2%, about 3%, about 4%, about 5%, about 6%, about 7%, about 8%, about 9%, about 10%, about 11%, about 12%, about 13%, about 14%, or about 15% to about 20%, about 21%, about 22%, about 23%, about 24%, about 25%, about 26%, about 27%, about 28%, about 29%, about 30%, about 31%, about 32%, about 33%, about 34%, about 35%, about 40%, about 50%, about 60%, about 70%, or about 80% (w/w) enteric material based on the weight of each delayed release gelling agent particle. In certain embodiments, the dosage form may contain from about 10% to about 30%, or from about 12% to about 25% (w/w) emetic material based on the weight of each delayed release gelling agent particle.

In certain embodiments, the delayed release gelling agent composition comprises rotor layered and enteric coated pellets, the pellets comprising from about 5% to about 35%, from about 5% to about 15%, from about 15% to about 25%, from about 25% to about 35%, about 10%, or about 30% (w/w) enteric material based on total weight of the delayed release gelling agent composition.

According to certain embodiments, the immediate release solid oral dosage forms disclosed herein may have a weight ratio of gelling agent to enteric material of from about 1:40 to about 40:1, from about 1:30 to about 30:1, from about 1:20 to about 20:1, from about 1:15 to about 15:1, from about 1:10 to about 10:1, from about 1:8 to about 8:1, from about 1:5 to about 5:1, from about 1:3 to about 3:1, from about 1:1.5 to about 1.5:1, from about 1:1 to about 40:1, from about 1:1 to about 30:1, from about 1:1 to about 20:1, from about 1:1 to about 15:1, from about 1:1 to about 8:1, from about 1:1 to about 5:1, from about 1:1 to about 3:1, from about 1:1 to about 1.5:1, from about 1:40 to about 1:1, from about 1:30 to about 1:1, from about 1:20 to about 1:1, from about 1:15 to about 1:1, from about 1:8 to about 1:1, from about 1:5 to about 1:1, from about 1:3 to about 1:1, or from about 1:1.5 to about 1:1.

### Aversive Agents

The immediate release solid oral dosage forms according to the disclosure may include one or more aversive agents to further deter illicit use of the active agent contained therein. Such aversive agents may be selected from the group consisting of emetics, antagonists, bittering agents, irritants, and mixtures thereof. The aversive agents may be incorporated into a matrix with the active agent (e.g., opioid analgesic) and delayed release gelling agent composition, or into particles, added separately within a capsule or as an additional tableting excipient.

Exemplary emetics include, but are not limited to, methyl cephaeline, cephaeline, emetine hydrochloride, psychotrine, O-methylpsychotrine, emetamine, ipecamine, hydro-ipecamine, ipecacunhic acid and mixtures thereof.

Exemplary antagonists include, but are not limited to, naltrexone, naloxone, nalmefene, cyclazacine, levallorphan, pharmaceutically acceptable salts thereof, solvates thereof, prodrugs thereof, and mixtures thereof.

Exemplary bittering agents include, but are not limited to, flavor oils, flavoring aromatics, oleoresins, plant extracts, leaf extracts, flower extracts, fruit extracts, sucrose derivatives, chlorosucrose derivatives, quinine sulphate, denatonium benzoate and mixtures thereof. In certain embodiments, the bittering agent may be selected from the group consisting of spearmint oil, peppermint oil, eucalyptus oil, oil of nutmeg, allspice, mace, oil of bitter almonds, menthol and mixtures thereof. In certain embodiments, the fruit extract may be from a fruit including, but not limited to lemon, orange, lime, grapefruit, and mixtures thereof.

Exemplary irritants include, but are not limited to, a surfactant, capsaicin or a capsaicin analog and mixtures thereof. The capsaicin analog may be selected from the group consisting of resiniferatoxin, tinyatoxin, heptanoylisobutylamide, heptanoyl guaiacylamide, an isobutylamide, a guaiacylamide, dihydrocapsaicin, homovanillyl octylester, nonanoyl vanillylamide, and mixtures thereof.

Exemplary surfactants include, but are not limited to, a poloxamer, a sorbitan monoester, a glyceryl monooleate, sodium lauryl sulfate and combinations thereof. In certain embodiments, the surfactant may be sodium lauryl sulfate.

In certain embodiments, the irritant may be included in the dosage form in an amount of, for example, from about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or about 10% to about 11%, about 12%, about 13%, about 14%, about 15%, about 16%, about 17%, about 18%, about 19%, about 20%, about 25%, about 30%, about 35%, about 40%, about 45%, or about 50% (w/w) irritant based on the weight of the dosage form. In certain embodiments, the dosage form may contain from about 0.1% to about 30%, from about 0.5% to about 20%, or from about 1% to about 10% (w/w) irritant based on the total weight of the dosage form.

### Excipients

The immediate release solid oral dosage forms according to the disclosure may include one or more pharmaceutically acceptable carriers and excipients. Examples of possible pharmaceutically acceptable carriers and excipients are described in the Handbook of Pharmaceutical Excipients, American Pharmaceutical Association (6th Edition, 2009 Publication), which is incorporated by reference herein. Suitable carriers and excipients include, but are not limited to, plasticizers, colorants, lubricants, fillers, thermal lubricants, antioxidants, buffering agents, disintegrants, binding agents, diluents, glidants, anti-adherants, sweeteners, chelating agents, flavorants, surfactants, solubilizers, stabilizers, hydrophilic polymers, hydrophobic polymers, waxes, lipophilic materials, absorption enhancers, preservative, absorbent, cross-linking agents, bioadhesive polymers, pore formers, osmotic agents, polycarboxylic acids or combinations thereof.

According to certain embodiments, the dosage forms may include a plasticizer. Plasticizers may interact with hydrophobic materials resulting in a lower viscosity of the mixture as compared to the mixture without the plasticizer when measured under the same conditions. Suitable plasticizers include, but are not limited to, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, low molecular weight poly(ethylene glycol), citrate ester-type plasticizers, triacetin, propylene glycol and glycerin. Such plasticizers may include ethylene glycol, 1,2-butylene glycol, 2,3-butylene glycol, styrene glycol, diethylene glycol, triethylene glycol, tetraethylene glycol and other poly(ethylene glycol) compounds, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutyl sebacate, acetyltributylcitrate, triethyl citrate, glyceryl monostearate, polysorbate 80, acetyl triethyl citrate, tributyl citrate and allyl glycolate, and mixtures thereof. In certain embodiments, the plasticizer may be in an amount of about 5% or less, or about 4% or less, or about 2% or less, or 0% (i.e., plasticizer free). An exemplary plasticizer may be a glyceryl monostearate based plasticizer such as PlasaACRYL® HTP 20 obtained from Evonik Industries.

According to certain embodiments, the dosage form may include a glidant. A glidant is an excipient that improves the flow characteristics of a compressible powder such as tablet ingredients or granules. In certain embodiments, the glidant is silicon dioxide. Two exemplary glidants are colloidal silicon dioxide (CAB-O-SIL®) and Quso (also known as Phila Quartz). The amount of glidant that can be used ranges from about 0.1 wt % to about 5 wt %.

Suitable diluents useful in the dosage forms according to the disclosure include, but are not limited to, lactose USP, lactose USP (anhydrous), lactose USP (spray dried), starch USP, directly compressible starch, mannitol USP, sorbitol, dextrose monohydrate, microcrystalline cellulose NF, dibasic calcium phosphate dihydrate NF, sucrose-based diluents, confectioner's sugar, monobasic calcium sulfate monohydrate, calcium sulfate dihydrate NF, calcium lactate trihydrate granular NF, dextrates NF (e.g., Emdex™), dextrose (e.g., Cerelose™), inositol, hydrolyzed cereal solids such as the Maltrons™ and Mor-Rex™, amylose, powdered cellulose (e.g., Elcema™), calcium carbonate, glycine, bentonite, polyvinylpyrrolidone, and the like. In certain embodiments, the dosage forms described herein can include the diluents in the range of about 0.1% to about 99%, or from about 10% to about 80%, or from about 15% to about 70%, of the total weight of the formulation.

Suitable lubricants include, but are not limited to, glyceryl behenate (Compritol™ 888), metallic stearates (e.g., magnesium, calcium and sodium stearates), stearic acid, hydrogenated vegetable oils (e.g., Sterotex™), talc, waxes such as beeswax and carnauba wax, silica, fumed silica, colloidal silica, calcium stearate, long chain fatty alcohols, boric acid, sodium benzoate and sodium acetate, sodium chloride, DL-Leucine, polyethylene glycols (e.g., Carbowax™ 4000 and Carbowax™ 6000), sodium oleate, sodium benzoate, sodium acetate, sodium lauryl sulfate, sodium stearyl fumarate (Pruv™), magnesium lauryl sulfate, stearic acid, stearyl alcohol, mineral oil, paraffin, micro crystalline cellulose, glycerin, propylene glycol and combinations thereof. In certain embodiments, the dosage forms may include one or more lubricants in an amount of from about 0.1% to about 15%, or from about 0.25% to about 10%, or from about 1% to about 8%, of the total weight of the dosage form. Magnesium stearate is a preferred lubricant for use in certain embodiments of the dosage forms.

Suitable anti-adherents include, but are not limited to, talc, cornstarch, colloidal silicone dioxide (Cab-O-Sil™), DL-Leucine, sodium lauryl sulfate, and metallic stearates. In certain implementations, the dosage forms can include an anti-adherent in an amount from about 0.1% to about 15%, or from about 0.25% to about 10%, or from about 1% to about 8%, of the total weight of the dosage form.

Other excipients (such as colorants, flavors and sweeteners) can be utilized in embodiments of the dosage forms where they impart little to no deleterious effect on the stability of the dosage form.

### Abuse Deterrence

### Extraction

In certain embodiments of the immediate release solid oral dosage forms described herein, recovery of the active agent from the dosage form may be less than about 40%, less than about 30%, less than about 20%, less than about 10%, less than about 8%, less than about 6%, less than about 4%, or less than about 2% based on a syringeability test whereby an intact solid oral dosage form is dissolved with 5 ml of solvent (such as tap water) with agitation at room temperature and the resultant solution is aspirated with a 18, 22, 25, or 27 gauge needle. In certain embodiments, the dissolution can be for a time from about 1 to about 60 minutes, e.g., about 5 minutes, about 10 minutes, about 15 minutes, about 30 minutes or about 45 minutes.

In certain embodiments, the recovery of the active agent from the dosage form may be less than about 40%, less than about 30%, less than about 20%, less than about 10%, less than about 8%, less than about 6%, less than about 4%, or less than about 2% based on a syringeability test whereby an intact solid oral dosage form is dissolved with 10 ml of solvent (such as tap water) with agitation at room temperature and the resultant solution is aspirated with a 18, 22, 25, or 27 gauge needle.

In certain embodiments, the recovery of the active agent from the dosage form may be less than about 40%, less than about 30%, less than about 20%, less than about 10%, less than about 8%, less than about 6%, less than about 4%, or less than about 2% based on a syringeability test whereby an intact solid oral dosage form is dissolved with 5 ml of solvent (such as tap water) without agitation at room temperature and the resultant solution is aspirated with a 18, 22, 25, or 27 gauge needle.

In certain embodiments, the recovery of the active agent from the dosage form may be less than about 40%, less than about 30%, less than about 20%, less than about 10%, less than about 8%, less than about 6%, less than about 4%, or less than about 2% based on a syringeability test whereby an intact solid oral dosage form is dissolved with 10 ml of solvent (such as tap water) without agitation at room temperature and the resultant solution is aspirated with a 18, 22, 25, or 27 gauge needle.

In certain embodiments, the recovery of the active agent from the dosage form may be less than about 40%, less than about 30%, less than about 20%, less than about 10%, less than about 8%, less than about 6%, less than about 4%, or less than about 2% based on a syringeability test whereby a crushed solid oral dosage form is dissolved with 10 ml of solvent (such as tap water) with agitation at room temperature and the resultant solution is aspirated with a 18, 22, 25, or 27 gauge needle.

In certain embodiments, the recovery of the active agent from the dosage form may be less than about 40%, less than about 30%, less than about 20%, less than about 10%, less than about 8%, less than about 6%, less than about 4%, or less than about 2% based on a syringeability test whereby a crushed solid oral dosage form is dissolved with 5 ml of solvent (such as tap water) with agitation at room temperature and the resultant solution is aspirated with a 18, 22, 25, or 27 gauge needle.

In certain embodiments, the recovery of the active agent from the dosage form may be less than about 40%, less than about 30%, less than about 20%, less than about 10%, less than about 8%, less than about 6%, less than about 4%, or less than about 2% based on a syringeability test whereby a crushed solid oral dosage form is dissolved with 10 ml of solvent (such as tap water) without agitation at room temperature and the resultant solution is aspirated with a 18, 22, 25, or 27 gauge needle.

In certain embodiments, the recovery of the active agent from the dosage form may be less than about 40%, less than about 30%, less than about 20%, less than about 10%, less than about 8%, less than about 6%, less than about 4%, or less than about 2% based on a syringeability test whereby a crushed solid oral dosage form is dissolved with 5 ml of solvent (such as tap water) without agitation at room temperature and the resultant solution is aspirated with a 18, 22, 25, or 27 gauge needle.

In certain embodiments, the ratio of the viscosity of a solution obtained from an intact solid oral dosage form at 5 minutes in about 5 ml water at room temperature to the viscosity of a solution obtained from an intact solid oral dosage form at 5 minutes in about 5 ml 0.1N HCl at room temperature is about 10:1 or more, about 15:1 or more, about 20:1 or more, about 25:1 or more, or about 30:1 or more.

### Disintegration

In certain embodiments, the intact immediate release solid oral dosage forms disclosed herein have a disintegration time in water ranging from about 10 second, 20 seconds, 30 seconds, 40 seconds, 50 seconds, or 1 minute to about 2 minutes, 3 minutes, 4 minutes, 5 minutes, 6 minutes, 7 minutes, 8 minutes, 9 minutes, 10 minutes, 15 minutes, 20 minutes, 25 minutes, or 30 minutes. In certain embodiments, the intact immediate release solid oral dosage forms disclosed herein have a disintegration time in water ranging from about 10 seconds to about 30 minutes, from about 1 minute to about 10 minutes, or from about 10 seconds to about 2 minutes.

In certain embodiments, the intact immediate release solid oral dosage forms disclosed herein have a disintegration time in Simulated Gastric Fluid (SGF) ranging from about 10 second, 20 seconds, 30 seconds, 40 seconds, 50 seconds, or 1 minute to about 2 minutes, 3 minutes, 4 minutes, 5 minutes, 6 minutes, 7 minutes, 8 minutes, 9 minutes, 10 minutes, 15 minutes, 20 minutes, 25 minutes, or 30 minutes. In certain embodiments, the intact immediate release solid oral dosage forms disclosed herein have a disintegration time in SGF ranging from about 10 seconds to about 30 minutes, from about 1 minute to about 10 minutes, or from about 10 seconds to about 2 minutes.

### Methods

### Methods of Treatment

The condition or disease states that can be treated by the dosage forms described herein include, but are not limited to, a condition or symptom that is inhibited, reduced or alleviated by opioid receptor activation. This refers to conditions or symptoms that are associated with one or more of the nervous system, vascular system, gastrointestinal system, pulmonary system and heart. Examples of such conditions are pain, pulmonary edema, cough and diarrhea.

According to various embodiments of the disclosure, also provided is a method of treating or preventing pain by administering a dosage form, for example, an immediate release solid oral dosage form as described herein, to a patient in need thereof. In embodiments, the dosage forms described herein can be used to treat or prevent acute or chronic pain. For example, the dosage forms can be used to treat or prevent pain, including, but not limited to, cancer pain, central pain, labor pain, myocardial infarction pain, pancreatic pain, colic pain, post operative pain, headache pain, muscle pain, and pain associated with intensive care.

The dosage forms described herein can also be used for treating or preventing pain associated with inflammation or with an inflammatory disease in a subject. The pain to be treated or prevented may be associated with inflammation associated with an inflammatory disease, which can arise where there is an inflammation of the body tissue, and which can be a local inflammatory response and/or a systemic inflammation. For example, the dosage forms can be used to treat or prevent pain associated with inflammatory diseases including, but not limited to: organ transplant rejection; reoxygenation injury resulting from organ transplantation (see Grupp et al., Protection against Hypoxia-reoxygenation in the Absence of Poly (ADP-ribose) Synthetase in Isolated Working Hearts, J. Mol. Cell Cardiol. 31:297 303 (1999)) including, but not limited to, transplantation of the heart, lung, liver, or kidney; chronic inflammatory diseases of the joints, including arthritis, rheumatoid arthritis, osteoarthritis and bone diseases associated with increased bone resorption; inflammatory bowel diseases, such as ileitis, ulcerative colitis, Barrett's syndrome, and Crohn's disease; inflammatory lung diseases, such as asthma, adult respiratory distress syndrome, and chronic obstructive airway disease; inflammatory diseases of the eye, including corneal dystrophy, trachoma, onchocerciasis, uveitis, sympathetic ophthalmitis and endophthalmitis; chronic inflammatory diseases of the gum, including gingivitis and periodontitis; tuberculosis; leprosy; inflammatory diseases of the kidney, including uremic complications, glomerulonephritis and nephrosis; inflammatory diseases of the skin, including sclerodermatitis, psoriasis and eczema; inflammatory diseases of the central nervous system, including chronic demyelinating diseases of the nervous system, multiple sclerosis, AIDS-related neurodegeneration and Alzheimer s disease, infectious meningitis, encephalomyelitis, Parkinson's disease, Huntington's disease, amyotrophic lateral sclerosis, and viral or autoimmune encephalitis; autoimmune diseases, including Type I and Type II diabetes mellitus; diabetic complications, including, but not limited to, diabetic cataract, glaucoma, retinopathy, nephropathy (such as microaluminuria and progressive diabetic nephropathy), polyneuropathy, mononeuropathies, autonomic neuropathy, gangrene of the feet, atherosclerotic coronary arterial disease, peripheral arterial disease, nonketotic hyperglycemic hyperosmolar coma, foot ulcers, joint problems, and a skin or mucous membrane complication (such as an infection, a shin spot, a candidal infection or necrobiosis lipoidica diabeticorum); immune-complex vasculitis, systemic lupus erythematosus (SLE); inflammatory diseases of the heart, such as cardiomyopathy, ischemic heart disease hypercholesterolemia, and atherosclerosis; as well as various other diseases that can have significant inflammatory components, including preeclampsia, chronic liver failure, brain and spinal cord trauma, and cancer. The dosage forms described herein can also be used for treating or preventing pain associated with inflammatory disease that can, for example, be a systemic inflammation of the body, exemplified by gram-positive or gram negative shock, hemorrhagic or anaphylactic shock, or shock induced by cancer chemotherapy in response to pro-inflammatory cytokines, e.g., shock associated with pro-inflammatory cytokines. Such shock can be induced, e.g., by a chemotherapeutic agent that is administered as a treatment for cancer.

### Methods of Preparation

In some embodiments, the present invention is directed to methods of preparing the immediate release solid oral dosage forms disclosed herein. Spheroids or beads coated with an active agent composition and/or with delayed release gelling agent composition may be prepared, for example, by rotor layering, by top spraying, by roller compacting, by bottom spraying (e.g., wurster coating), or by any conceivable combination of these methods. In certain embodiments, spheroids or beads coated with an active agent composition and/or with delayed release gelling agent composition may be prepared by roller compacting followed by milling and sieving.

In certain embodiments, a process for preparing immediate release solid oral dosage forms according to the invention comprise (i) preparing a delayed release gelling agent composition; (ii) blending the delayed release gelling agent composition with an active agent composition; and (iii) compressing the blend into a tablet. In certain embodiments, preparing the delayed release gelling agent composition may comprise enteric coating a gelling agent.

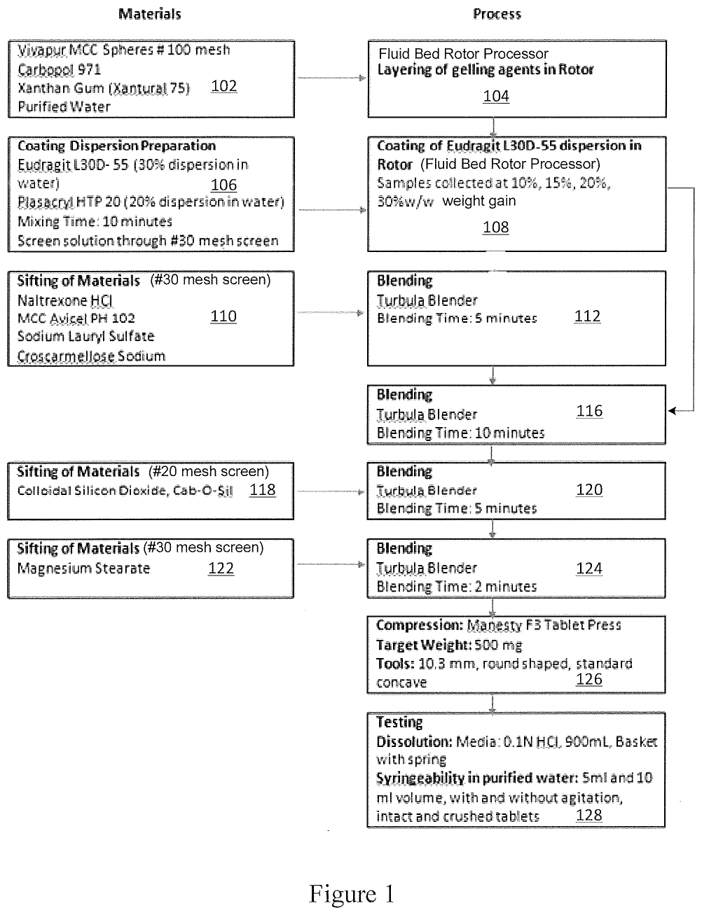

For instance, FIG. 1 presents a non-limiting process flow diagram for the manufacture of an immediate release solid oral dosage form by the rotor layering method. In blocks 102 and 104 inert beads or cores (e.g., microcrystalline cellulose spheres, MCC) are coated with gelling agents (e.g., carbopol 971 and xanthan gum in water). In block 106, a coating dispersion comprising the enteric material (e.g., Eudragit® L30D-55) and plasticizer (e.g. PlasaACRYL™ HTP20) are mixed and screened through a #30 mesh screen. In block 108, the coating dispersion from block 106 is rotor layered onto the coated gelling agents from block 104 to form rotor layered and enteric coated delayed release gelling agent composition. Blocks 110 through 126 describe the preparation of a compressed Naltrexone tablet by blending the various components (Naltrexone HCl, MCC, sodium lauryl sulfate, and croscarmellose in block 112, rotor layered and enteric coated delayed release gelling agent composition in block 116, colloidal silicon dioxide in block 120, and magnesium stearate in block 124) and ultimately compressing the blend from block 124 to form a compressed tablet in block 126. The compressed tablet of block 126 may undergo dissolution and syringeability testing as described in the examples below.

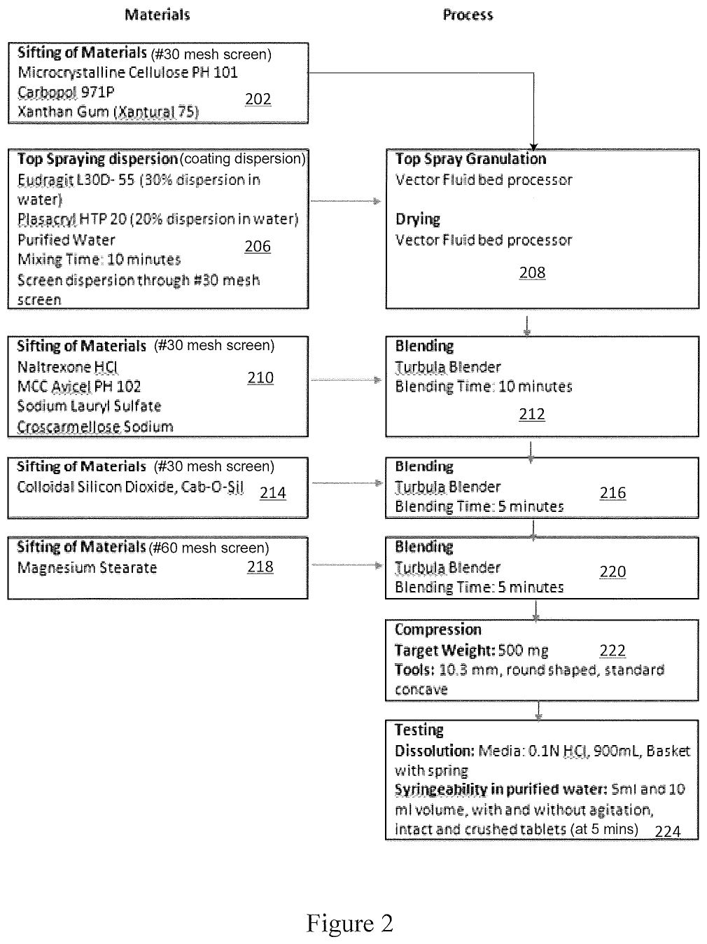

Another example of a non-limiting process for preparing an immediate release solid oral dosage form according to the present invention is illustrated in FIG. 2. FIG. 2 presents a process flow diagram for the manufacture of an immediate release solid oral dosage form by the top spray granulation method. In blocks 202 through 208 inert beads or cores (e.g, microcrystalline cellulose), gelling agents (e.g., Carbopol 971 and xanthan gum), enteric material dispersion in water (e.g., Eudragit® L30D-55) and plasticizer dispersion in water (e.g. PlasaACRYL HTP20) undergo top spray granulation in a Vector Fluid bed processor. Blocks 210 through 222 describe the preparation of a compressed Naltrexone tablet by blending the various components (Naltrexone HCl, MCC, sodium lauryl sulfate, croscarmellose, and top spray delayed release gelling agent granules in block 212, colloidal silicon dioxide in block 216, and magnesium stearate in block 220) and ultimately compressing the blend from block 220 to form a compressed tablet in block 222. The compressed tablet of block 222 may undergo dissolution and syringeability testing, in accordance with block 224, as described in the examples below.

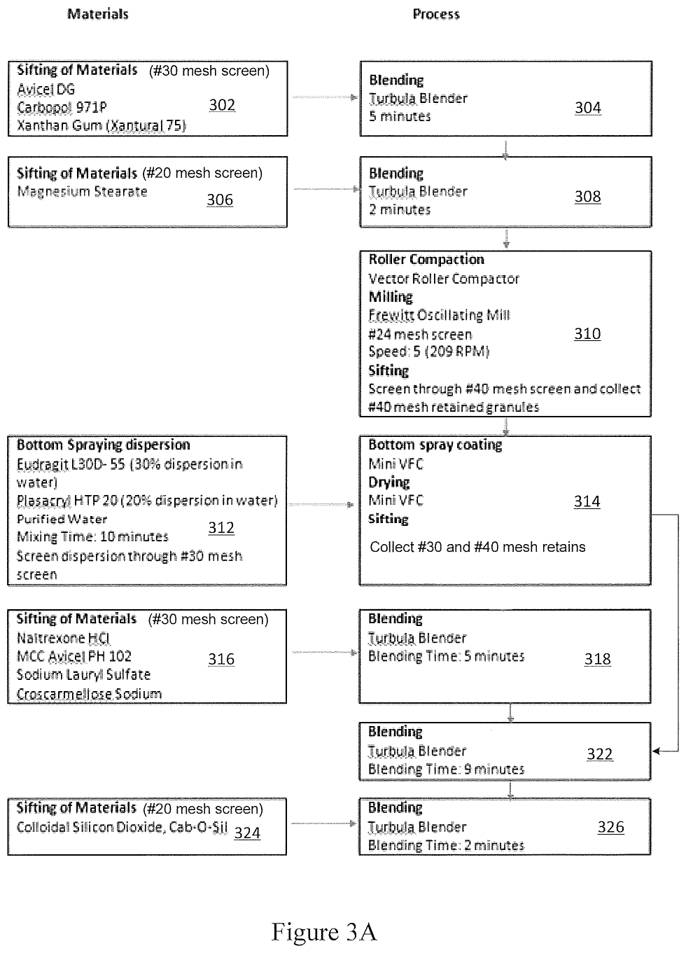

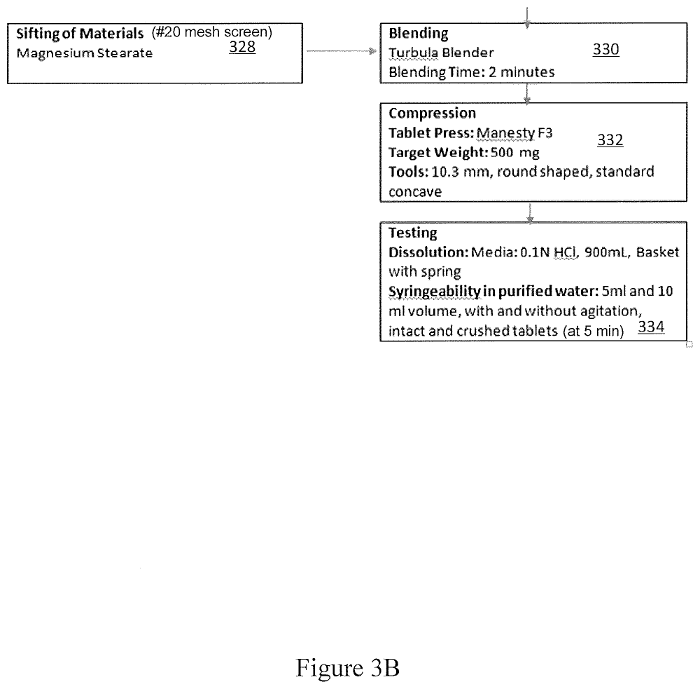

Another example of a non-limiting process for preparing an immediate release solid oral dosage form according to the present invention is illustrated in FIGS. 3A-3B. FIGS. 3A-3B present a process flow diagram for the manufacture of an immediate release solid oral dosage form by the roller compaction/milling/sifting and bottom spray (wurster coating) method. In blocks 302 through 314 inert beads or cores (e.g, microcrystalline cellulose spheres, MCC), gelling agents (e.g., carbopol 971 and xanthan gum), magnesium stearate, enteric material dispersion in water (e.g., Eudragit® L30D-55) and plasticizer dispersion in water (e.g. PlasaACRYL™ HTP20) undergo roller compaction, milling, sifting, and bottom spray coating. Blocks 316 through 332 describe the preparation of a compressed Naltrexone tablet by blending the various components (Naltrexone HCl, MCC, sodium lauryl sulfate, and croscarmellose in block 318, bottom sprayed and roller compacted delayed release gelling agent granules in block 322, colloidal silicon dioxide in block 326, and magnesium stearate in block 330 and ultimately compressing the blend from block 330 to form a compressed tablet in block 332. The compressed tablet of block 332 may undergo dissolution and syringeability testing, in accordance with block 334, as described in the examples below.

In certain embodiments, the process for preparing an immediate release solid oral dosage form may comprise (i) preparing one or more particles; (ii) coating the one or more particles with an opioid analgesic composition; and (iii) blending the one or more particles coated with opioid analgesic composition with a delayed release gelling agent composition, wherein the solid oral dosage form comprises an immediate release opioid analgesic composition, and wherein the solid oral dosage form is free of or substantially free of an extended release opioid analgesic composition. The process may further comprise (iv) compressing the blend into a tablet.

In certain embodiments, the process for preparing an immediate release solid oral dosage form may comprise (i) preparing one or more delayed release gelling agent particles; and (ii) coating the one or more delayed release gelling agent particles with an opioid analgesic composition, wherein the solid oral dosage form comprises an immediate release opioid analgesic composition, and wherein the solid oral dosage form is free of or substantially free of an extended release opioid analgesic composition. The process may further comprise (iii) compressing the one or more coated particles into a tablet.

In certain embodiments, the process for preparing an immediate release solid oral dosage form may comprise (i) preparing one or more delayed release gelling agent particles; and (ii) dispersing an opioid analgesic composition and the one or more delayed release gelling agent particles in a matrix, wherein the solid oral dosage form comprises an immediate release opioid analgesic composition, and wherein the solid oral dosage form is free of or substantially free of an extended release opioid analgesic composition.

### EXAMPLES

The following examples are set forth to assist in understanding the invention and should not, of course, be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### Example 1: Enteric Coated Pellets Prepared Through a Rotor Layering Process

In this example, a gelling agent was layered on microcrystalline cellulose (MCC) spheres via rotor layering. Subsequently, an enteric coating was layered onto the pellets.

TABLE 1
Rotor Layered Pellets

| | % w/w | mg/Tablet |
|---|---|---|
| Layering of Gelling agents | | |
| Vivapur ® # 100 mesh | 66.05 | 91.000 |
| Carbopol ® 971P | 8.71 | 12.000 |
| Xantural ® 75 | 2.61 | 3.600 |
| Purified Water* | | q.s. |
| Enteric Coating of pellets | | |
| Eudragit ® L30D-55, Solids^{#} | 19.34 | 26.650 |
| PlasACRYL ™ HTP20, Solids^{#} | 3.29 | 4.531 |
| Total Enteric Coated Pellets | 100.00 | 137.781 |

*Used as a coating vehicle, evaporates during process.
^{#}Eudragit ® L 30D-55 is a 30% w/w dispersion, PlasACRYL ™ HTP20 is 20% w/w dispersion.
Vivapur ®—microcrystalline cellulose spheres
Carbopol ® 971P—carboxypolymethylene or carbomer
Xantural ® 75—xanthan gum
Eudragit ® L30-55—methacrylic acid—ethyl acrylate copolymer (1:1)
PlasACRYL ™ HTP20—aqueous dispersion containing an anti-tacking agent, a plasticizer, and a stabilizer

### Layering of Gelling Agents Procedure

Carbopol® 971P and Xantural® 75 (Xanthan Gum) were sifted together through #30 mesh screen and mixed in a polybag for 5 minutes to form a powder blend. Vivapur® spheres were charged in rotor insert of VFC fluid bed equipment. The carbopol and xanthan gum powder blend was fed to the rotor using K-Tron powder feeder. The entire quantity of powder blend was layered on the MCC spheres to form powder layered pellets. The powder layered pellets were dried for 30 minutes in the rotor. The process parameters for powder layering are disclosed in Table 2 below.

TABLE 2
Process Parameters for Powder Layering of Gelling Agents

| | | | | | | | | | Pressure Drop | |
|---|---|---|---|---|---|---|---|---|---|---|
| Solution | Blend | Rotor | Slit Air | | Product | Fluid Bed Air | | | Slit | Filter |
| Rate | Rate | Speed, | Volume | Temp. | Temp. | Inlet | Exhaust | Vol | Air | Air |
| (gpm) | (gpm) | rpm | (cfm) | (° C.) | (° C.) | Temp. | Temp. | (cfm) | ″WC | ″WC |

Powder Layering Process, Total Time: 100 Minutes

| | | | | | | | |
|---|---|---|---|---|---|---|---|
| 2.7 | 1.2 | 250 | 5 | 39.5-40.1 | 18.7-19.9 | 0.0 | 1.8-2.2 |

Drying Process, Total Time: 30 Minutes

| | | | | | | | | |
|---|---|---|---|---|---|---|---|---|
| 150 | 5 | 40.0 | 22.3-32.3 | 31.8-41.0 | 21.8-30.9 | 53-54 | 0.0 | 1.8-2.0 |

### Layering of Enteric Coating Procedure

An aqueous dispersion of Eudragit® L30D-55 was prepared by adding purified water and PlasACRYL™ HTP20 to Eudragit® L30D-55 while stirring with a propeller stirrer. The dispersion was mixed for at least 10 minutes. Dried powder layered pellets were coated with the enteric coating dispersion in a rotor process to form coated pellets. The coated pellets were dried for 20 minutes to form Rotor Layered Pellets. The process parameters for enteric coating layering are disclosed in Table 3 below.

TABLE 3
Process Parameters for Enteric Coating Layering

| Solution | Blend | Rotor | Slit Air | | Product | Fluid Bed Air | | |
|---|---|---|---|---|---|---|---|---|
| Rate | Rate | Speed, | Volume | Temp. | Temp. | Inlet | Exhaust | Vol |
| (gpm) | (gpm) | rpm | (cfm) | (° C.) | (° C.) | Temp. | Temp. | (cfm) |

Powder Layering Process, Total Time: 172 Minutes

| | | | | |
|---|---|---|---|---|
| 3.5-3.9 | 150-300 | 5 | 31.8-32.0 | 18.4-19.8 |

Drying Process, Total Time: 20 Minutes

| | | | | | | |
|---|---|---|---|---|---|---|
| 100-150 | 5 | 32.0 | 22.8-27.3 | 27.9-31.7 | 21.5-26.1 | 56 |

Samples of enteric coated pellets were withdrawn at 10%, 15%, 20% and 30% of target enteric coating weight gain.

### Example 2: Naltrexone Tablets Comprising Rotor Layered Pellets of Example 1

Naltrexone tablets were made in two lots using Rotor Layered Pellets from Example 1 (Lot 2428-053A and Lot 2428-053B) as summarized in Table 4 below and in FIG. 1. Lot 2428-053A had a higher content of gelling agents (carbopol 30 mg, xanthan gum 9 mg) and 10% enteric coating. Lot 2428-053B had a lower content of gelling agents (carbopol 25 mg, xanthan gum 7.5 mg) and 30% enteric coating. The percent enteric coating was calculated based on weight gain obtained from adding the Eudragit® L-30D-55 and PlasACRYL™ HTP 20 to the gelling agent composition comprising the MCC spheres, carbopol, and xanthan gum.

TABLE 4
Formulation for Naltrexone Tablets
Containing Rotor Layered Pellets

| | High Gelling Polymer, | | Low Gelling Polymer, | |
|---|---|---|---|---|
| | 10% Enteric Coating | | 30% Enteric Coating | |
| | Lot No. | | | |
| | Lot 2428-053A | | Lot 2428-053B | |
| | | mg/ | | mg/ |
| | % w/w | Tablet | % w/w | Tablet |
| Rotor Layered Pellets in | | | | |
| Tablet | | | | |
| Naltrexone HCl | 6.00 | 30.000 | 6.00 | 30.000 |
| Avicel ® PH 102 | 19.50 | 97.515 | 20.60 | 103.000 |
| Sodium Lauryl Sulfate | 6.00 | 30.000 | 6.00 | 30.000 |
| Ac-Di-Sol ® | 9.00 | 45.000 | 9.00 | 45.000 |
| Rotor Layered Pellets of | | | | |
| Example 1 | | | | |
| Vivapur ® MCC | 45.50 | 227.500 | 37.91 | 189.551 |
| spheres, # 100 mesh | | | | |
| Carbopol ® 971P | 6.00 | 30.000 | 5.00 | 24.996 |
| Xantural ® 75 | 1.80 | 9.000 | 1.50 | 7.499 |
| Eudragit ® L-30D-55, | 4.44 | 22.210 | 11.10 | 55.512 |
| Solids | | | | |
| PlasACRYL ™ HTP20, | 0.76 | 3.775 | 1.89 | 9.438 |
| Solids | | | | |
| Cab-O-Sil ® | 0.50 | 2.500 | 0.50 | 2.500 |
| Magnesium Stearate | 0.50 | 2.500 | 0.50 | 2.500 |
| Total Tablet Weight | 100.00 | 500.00 | 100.00 | 500.00 |

Avicel ® PH 102—microcrystalline cellulose
Ac-Di-Sol ®—croscarmellose sodium
Cab-O-Sil ®—colloidal silicon dioxide

### Procedure:

1) Sift through #30 mesh screen and blend for 5 minutes Naltrexone HCl, Microcrystalline Cellulose Avicel® PH 102, Sodium Lauryl Sulfate, Croscarmellose Sodium (Ac-Di-Sol®).

2) Add Rotor Layered Pellets from Example 1 to the blend from step 1 and blend for ten minutes.

3) Sift Colloidal Silicon Dioxide through #20 mesh screen and then add to blend from step 2 and blend for five minutes.

4) Sift Magnesium Stearate through #20 mesh screen and then add to blend from step 3 and blend for two minutes.

5) Compression: compress mixture from step 4 through tablet press in accordance with the parameters of Table 5.

TABLE 5
Compression Process Parameters

| | Lot 2428-053A | Lot 2428-053B | Lot 2428-053B |
|---|---|---|---|
| Equipment Used | Manesty F3 Tablet Press | | |
| Tooling | 10.3 mm, round shaped, standard concave | | |
| Weight, mg | 500 | 500 | 500 |
| Thickness, inches | 0.2235 | 0.2225 | 0.2140 |
| Hardness, kp | 5 kp | 5 kp | 9 kp |
| Disintegration | Not performed | 2 min 5 sec | 8 min 5 sec |
| Time, SGF | due to insufficient | | |
| Disintegration | number of tablets | 2 min 30 sec | 7 min 45 sec |
| Time, Water | | | |

### Example 3: Enteric Coated Granules Prepared Through a Top Spray Fluid Bed Granulation Process

In this example, enteric granules of gelling agents were prepared by top spray granulation.

TABLE 6
Top Spray Enteric Granules of Gelling Agent

| Top Spray Granulation | % w/w | mg/tab |
|---|---|---|
| Microcrystalline Cellulose PH 101 | 55.86 | 120.000 |
| Carbopol ® 971P | 13.97 | 30.000 |
| Xantural ® 75 (Xanthan Gum) | 4.19 | 9.000 |
| Eudragit ® L30D-55, Solids^{#} | 22.21 | 47.700 |
| PlasACRYL ™ HTP20, Solids^{#} | 3.77 | 8.109 |
| Purified Water, USP* | | q.s. |
| Total enteric granules of gelling agents | 100.00 | 214.81 |

*Used as a granulating agent, evaporates during process.
^{#}Eudragit ® L 30D-55 is a 30% w/w dispersion, PlasACRYL ™ HTP 20 is 20% w/w dispersion.

Microcrystalline Cellulose PH 101, Carbopol® 971P and Xantural® 75 (Xanthan Gum) were sifted together through #30 mesh screen and collected and loaded into a Fluid Bed Granulator bowl. Purified Water and PlasACRYL™ HTP20 were added to Eudragit® L30D-55 while stirring with a propeller stirrer to form a dispersion. The dispersion was mixed for at least 10 minutes. The dispersion was screened through #30 mesh screen and was sprayed onto a bed of material using top spray gun assembly in accordance with the process parameters summarized in Table 7 below.

TABLE 7
Top Spray Granulation Process Parameters

| | | | | |
|---|---|---|---|---|
| Inlet | Product | Air | Spray | |
| Temp. | Temp. | Volume, | rate, | Atomizing Air |
| ° C. | ° C. | CFM | g/min | Pressure, psi |

Top Spray Granulation Parameters, Total Time: 6.5 minutes

| | | | | |
|---|---|---|---|---|
| 25.9-31.1 | 23.8-26.4 | 29-50 | 4 | 30-35 |

Drying Parameters, Total Time: 30 minutes

| | | |
|---|---|---|
| 43.2-46.2 | 32.3-35.1 | 43-44 |

The resulting enteric granules were screened through a #60, #80, #100, and a #200 mesh screens. There were some agglomerates formed during the process which were rejected. Granules retained on #60 and #80 mesh screens were rejected as well. Tablets were made using composite granules of retains of #100 mesh, #200 mesh and Pan. The percentage of retained granules after sifting through the various mesh screens is summarized in Table 8 below.

TABLE 8
Particle Size Analysis

| Screen Size | % Retained |
|---|---|
| # 60 | 0.37 |
| # 80 | 0.37 |
| #100 | 39.74 |
| #200 | 38.99 |
| Pan | 20.54 |

### Example 4: Naltrexone Tablets Comprising Top Sprayed Granules of Example 3

Naltrexone tablets were made in two lots using Top Sprayed Granules from Example 3 (Lot 2428-048A and Lot 2428-048B) as summarized in Table 9 below and in FIG. 2. Lot 2428-048A had a higher content of gelling agents (carbopol 30 mg, xanthan gum 9 mg). Lot 2428-048B had a lower content of gelling agents (carbopol 25 mg, xanthan gum 7.5 mg).

TABLE 9
Formulation for Naltrexone Tablets
Containing Top Sprayed Granules

| | 2428-048A High | | 2428-048B Low | |
|---|---|---|---|---|
| | Gelling Polymer | | Gelling Polymer | |
| | | mg/ | | mg/ |
| | % w/w | Tablet | % w/w | Tablet |
| Top Sprayed Granules in Tablet | | | | |
| Naltrexone HCl | 6.00 | 30.000 | 6.00 | 30.000 |
| Avicel ® PH 102 | 39.64 | 198.191 | 52.60 | 263.000 |
| Sodium Lauryl Sulfate | 6.00 | 30.000 | 6.00 | 30.000 |
| Croscarmellose Sodium | 4.40 | 22.000 | 4.40 | 22.000 |
| Top Sprayed Granules of | | | | |
| Example 3 | | | | |
| Avicel ® PH 101 | 24.00 | 120.000 | 16.76 | 83.795 |
| Carbopol ® 971P | 6.00 | 30.000 | 4.19 | 20.949 |
| Xantural ® 75 | 1.80 | 9.000 | 1.26 | 6.285 |
| Eudragit ® L-30D-55, Solids | 9.54 | 47.700 | 6.66 | 33.309 |
| PlasACRYL ™ HTP20, Solids | 1.62 | 8.109 | 1.13 | 5.662 |
| Cab-O-Sil ® | 0.50 | 2.500 | 0.50 | 2.500 |
| Magnesium Stearate | 0.50 | 2.500 | 0.50 | 2.500 |
| Total Tablet Weight | 100.00 | 500.00 | 100.00 | 500.00 |

Avicel ® PH 101—microcrystalline cellulose

### Procedure:

1) Sift Naltrexone HCl, Microcrystalline Cellulose Avicel® PH 102, Sodium Lauryl Sulfate, Croscarmellose Sodium (Ac-Di-Sol®) and Top Spray Granules from Example 3 through #30 mesh screen and blend for 10 minutes.

2) Sift Colloidal Silicon Dioxide through #30 mesh screen and add to the blend from step 1 and blend for 5 minutes.

3) Sift Magnesium Stearate through #60 mesh screen and add to the blend from step 2 and blend for 5 minutes.

4) Compression: Compress mixture from step 3 through tablet press in accordance with the parameters of Table 10.

TABLE 10
Compression Process Parameters

| | 2428-048A | | 2428-048A | | 2428-048B | | 2428-048B | |
|---|---|---|---|---|---|---|---|---|
| Equipment | Manesty F3 | | | | | | | |
| Used | | | | | | | | |
| Tooling | 10.3 mm round shaped, standard concave | | | | | | | |
| Weight, mg | 500 | | 500 | | 500 | | 500 | |
| Thickness, | 0.2600 | | 0.2400 | | 0.2600 | | 0.2400 | |
| inches | | | | | | | | |
| Hardness, kp | 10 | kp | 17 | kp | 10 | kp | 17 | kp |
| Disintegration | 1 | min | 1.5 | min | 0.5 | min | 1 | min |
| Time, SGF | | | | | | | | |
| Disintegration | >10 | min | >10 | min | 1 | min | 1.5 | min |
| Time, Water | | | | | | | | |

### Example 5: Particles Prepared Through Roller Compaction

In examples 5 through 6, gelling agent particles were prepared through roller compaction followed by wurster coating (i.e., bottom spray fluid bed coating).

TABLE 11
Roller Compaction Formulation

| Formula Ingredients | % w/w | mg/tab, solids |
|---|---|---|
| Avicel ® DG | 65.087 | 74.850 |
| Carbopol ® 971P | 26.087 | 30.000 |
| Xantural ® 75 | 7.826 | 9.000 |
| Magnesium Stearate | 1.000 | 1.150 |
| Total | 100.000 | 115.000 |

Avicel ® DG—microcrystalline cellulose

### Procedure:

1) Sift Avicel® DG, Carbopol® 971P and Xantural® 75through #30 mesh screen and blend for 10 minutes using Turbula blender.

2) Sift Magnesium Stearate through #20 mesh screen and add to the blend from step 1 and then blend for two minutes.

3) Use blend from step 2 for roller compaction in accordance with the roller compaction parameters summarized in Table 12.

TABLE 12
Roller Compaction Parameters

| Trial No. | Roller Speed, RPM | Auger Speed, RPM | Roll pressure, PSI |
|---|---|---|---|
| 1 | 3.3 | 18 | 800 |
| 2 | 3.3 | 18 | 1200 |
| 3 | 3.3 | 18 | 1500 |
| 4 | 2.3 | 23 | 1200 |
| 5 | 2.3 | 23 | 800 |
| 6 | 2.3 | 23 | 1500 |

| | |
|---|---|
| Milling | Frewitt Mill, Milling Screen #24 mesh, Speed 5 (209 rpm) |

The resulting particles were analyzed and their particle size distribution, bulk density, and tapped density are summarized in Table 13 below.

TABLE 13
Particle Size Distribution, Bulk and Tapped Density

| | % Retained | | | | | |
|---|---|---|---|---|---|---|
| Screen Size | Trial 1 | Trial 2 | Trial 3 | Trial 4 | Trial 5 | Trial 6 |
| 40 | 18.07 | 18.40 | 21.25 | 33.01 | 37.20 | 36.00 |
| 60 | 17.41 | 16.96 | 16.23 | 18.90 | 19.30 | 19.26 |
| 80 | 16.96 | 13.50 | 16.99 | 8.97 | 9.10 | 9.38 |
| 100 | 12.21 | 14.37 | 13.66 | 7.79 | 8.33 | 8.21 |
| 200 | 27.24 | 29.27 | 29.09 | 19.49 | 13.38 | 14.81 |
| 325 | 7.71 | 6.59 | 2.69 | 10.70 | 11.69 | 10.92 |
| Pan | 0.41 | 0.91 | 0.08 | 1.14 | 1.17 | 1.42 |
| Bulk Density, | 0.45 | 0.42 | 0.44 | 0.51 | 0.52 | 0.52 |
| g/ml | | | | | | |
| Tapped | 0.58 | 0.56 | 0.56 | 0.61 | 0.61 | 0.61 |
| Density, g/ml | | | | | | |

From the roller compaction trials, it was found that the roller speed and auger speed had higher impact on the particle size and density of the granules. Granules from Trial 6 were coated with Eudragit® L30D-55 dispersion as described in Example 6 below.

### Example 6: Fluid Bed Coating (Bottom Spray) of Roller Compacted Granules

In this example, the roller compacted particles from Example 5 underwent Wurster coating as summarized below.

TABLE 14
Wurster Coating Formulation

| Bottom Spray Coating Formula | % w/w | mg/tab, solids |
|---|---|---|
| Roller compacted granules from | 83.09 | 115.000 |
| Example 5 (#40 mesh retained) | | |
| Eudragit ® L30D-55^{#} | 14.45 | 20.000 |
| PlasACRYL ™ HTP 20^{#} | 2.46 | 3.400 |
| Purified Water* | | |
| Total | 100.00 | 138.400 |

*Used as a coating vehicle, evaporates during process.
^{#}Eudragit ® L30D-55 is a 30% w/w dispersion, PlasACRYL ™ HTP 20 is 20% w/w dispersion.

### Procedure:

1) Sift through #30 and #40 mesh screens granules from Trial 6 (Example 5).

2) Separate the fraction retained in the #40 mesh screen to be enteric coated with Eudragit® L30D-55.

3) Prepare a coating dispersion by adding Purified Water and PlasACRYL™ HTP20 to Eudragit® L30D-55 while stirring for at least 10 minutes using propeller stirrer.

4) Pass the dispersion through #30 mesh screen.

5) Spray the dispersion onto the granules from step 1 using bottom spray Wurster insert in Mini VFC in accordance with the Wurster coating parameters summarized in Table 15 below.

TABLE 15
Wurster Coating Parameters

| | | | | |
|---|---|---|---|---|
| Inlet | Exhaust | Air | Spray | |
| Temp. | Temp. | Volume, | Rate, | Atomizing Air |
| ° C. | ° C. | LPM | g/min | Pressure, psi |

Bottom Spray Granulation Parameters, Total Time: 33 minutes

| | | | | |
|---|---|---|---|---|
| 49.0-50.0 | 30.9-31.2 | 242-282 | (0.646 g/min) | 6.0-6.1 |

Drying Parameters, Total Time: 15 minutes

| | | |
|---|---|---|
| 50.0 | 36.0 | 350 |

Coated granules were screened through #20 mesh, #30 mesh and #40 mesh screens. Portion above #20 mesh was agglomerates that were discarded. The Portion retained on #30 and #40 mesh screens, combined, were used to make the tablets of Example 7.

### Example 7: Naltrexone Tablets Comprising Roller Compacted and Bottom Sprayed Granules of Example 6

Naltrexone tablets were made in two lots using Roller Compacted and Bottom Sprayed Granules from Example 6 (Lot 2428-069A and Lot 2428-069B) as summarized in Table 16 below and in FIGS. 3A-3B. Lot 2428-069A had a higher content of gelling agents (carbopol 30 mg, xanthan gum 9 mg). Lot 2428-069B had a lower content of gelling agents (carbopol 20 mg, xanthan gum 6 mg).

TABLE 16
Formulation for Naltrexone Tablets Containing
Roller Compacted and Bottom Sprayed Granules

| | 2428-069A High | | 2428-069B Low | |
|---|---|---|---|---|
| | Gelling Polymer | | Gelling Polymer | |
| | | mg/ | | mg/ |
| | % w/w | Tablet | % w/w | Tablet |
| Bottom Sprayed Granules in Tablet | | | | |
| Naltrexone HCl | 6.00 | 30.000 | 6.00 | 30.000 |
| Avicel ® PH 102 | 50.32 | 251.600 | 59.55 | 297.733 |
| Sodium Lauryl Sulfate | 6.00 | 30.000 | 6.00 | 30.000 |
| Croscarmellose Sodium | 9.00 | 45.000 | 9.00 | 45.000 |
| Bottom Sprayed Granules of | | | | |
| Example 6 | | | | |
| Avicel ® DG | | 74.850 | | 49.900 |
| Carbopol ® 971P | | 30.000 | | 20.000 |
| Xantural ® 75 | | 9.000 | | 6.000 |
| Magnesium Stearate | | 1.150 | | 0.767 |
| Eudragit ® L-30D-55, Solids | | 20.000 | | 13.333 |
| PlasACRYL ™ HTP 20, Solids | | 3.400 | | 2.267 |
| Cab-O-Sil ® | 0.50 | 2.500 | 0.50 | 2.500 |
| Magnesium Stearate | 0.50 | 2.500 | 0.50 | 2.500 |
| Total Tablet Weight | 100.00 | 500.000 | 100.00 | 500.000 |

### Procedure:

1) Sift Naltrexone HCl, Microcrystalline Cellulose Avicel® PH 102, Sodium Lauryl Sulfate, Croscarmellose Sodium (Ac-Di-Sol®). through #30 mesh screen and blend for 5 minutes

2) Add roller compacted and bottom sprayed granules of Example 6 into the mixture of step 1 and blend for nine minutes.

3) Sift Colloidal Silicon Dioxide through #20 mesh screen and add, to the blend from step 2 and blend for 2 minutes.

4) Sift Magnesium Stearate through #20 mesh screen and add to the blend from step 3 and blend for 2 minutes.

5) Compression: compress mixture from step 4 through tablet press in accordance with the parameters of Table 17.

TABLE 17
Compression Parameters

| | 2428-069A | | 2428-069A | | 2428-069B | | 2428-069B | |
|---|---|---|---|---|---|---|---|---|
| Equipment | Manesty F3 | | | | | | | |
| Used | | | | | | | | |
| Tooling | 10.3 mm round shaped, standard concave | | | | | | | |
| Weight, mg | 500 | | 500 | | 500 | | 500 | |
| Thickness, | 0.2500 | | 0.2290 | | 0.2665 | | 0.245 | |
| inches | | | | | | | | |
| Hardness, kp | 7 | kp | 15 | kp | 7 | kp | 15 | kp |
| Disintegration | 15 | sec | 2.5 | min | 15 | sec | 15 | sec |
| Time, SGF | | | | | | | | |
| Disintegration | 30 | sec | 2.5 | min | 21 | sec | 35 | sec |
| Time, Water | | | | | | | | |

The tablets of Examples 2, 4 and 7 underwent dissolution testing and syringeability testing. The testing procedure and results are summarized in Examples 8 and 9 and the corresponding figures.

### Example 8: Syringeability Testing

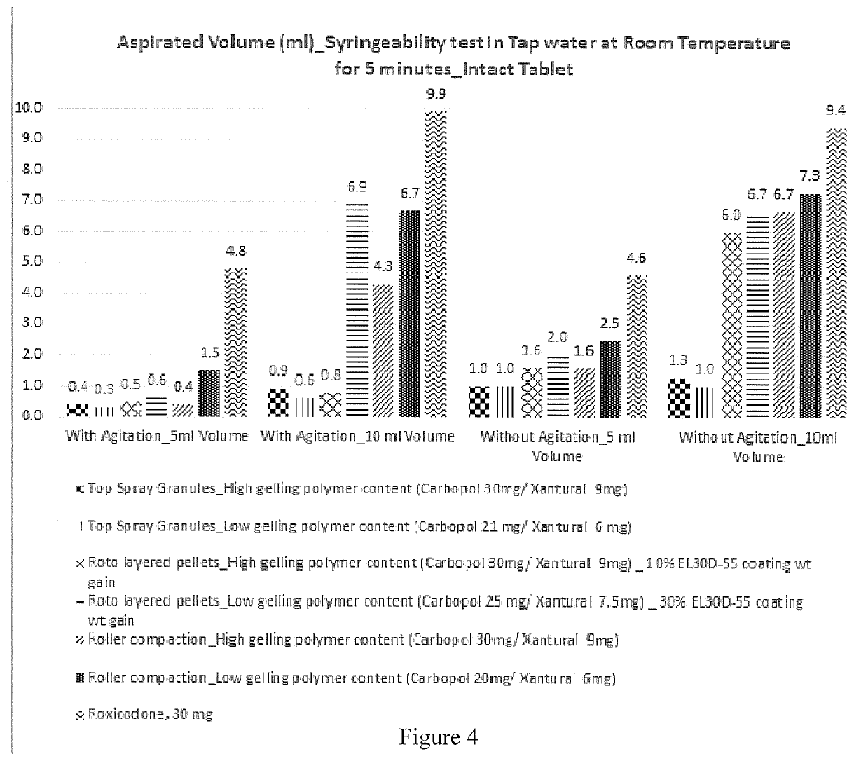

The tablets of Examples 2, 4 and 7 were tested for syringeability over five minutes post dissolution using 5 ml and 10 ml tap water at room temperature. Tablets were tested in intact and crushed form. Aspirated volume (over five minutes) was noted and was assayed for Naltrexone HCl content. Data is summarized in Table 18 to 23 and FIGS. 4 through 7.

TABLE 18
Volume (ml) Aspirated After Dissolution of Tablets in Tap Water at Room Temperature (Intact Tablet at 5 minutes - FIG. 4)

| | | | Rotor layered pellets | | | | |
|---|---|---|---|---|---|---|---|
| | | | (Example 2) | | | | |
| | Top Spray Granules | | Carbopol 30 mg/ | Carbopol/25 mg/ | Roller compaction | | |
| | (Example 4) | | Xantural 9 mg | Xantural 7.5 mg | (Example 7) | | Control |
| | Carbopol 30 mg/ | Carbopol 21 mg | 10% EL30D-55 | 30% EL30D-55 | Carbopol 30 mg/ | Carbopol 20 mg/ | Roxicodone, |
| | Xantural 9 mg | Xantural 6 mg | coating wt gain | coating wt gain | Xantural 9 mg | Xantural 6 mg | 30 mg |
| | N = 2, Average (min-max) | | | | | | |
| With | 0.4 (0.4-0.4) | 0.3 (0.2-0.3) | 0.5 (0.5-0.5) | 0.5 (0.4-0.5) | 0.4 (0.4-0.4) | 1.5 (1.4-1.6) | 4.8 (4.8-4.8) |
| Agitation_5 ml | | | | | | | |
| Volume | | | | | | | |
| With | 0.9 (0.8-0.9) | 0.6 (0.5-0.6) | 0.8 | 6.9 (6.8-7.0) | 4.3 (2.8-5.5) | 6.7 (6.4-7.0) | 9.9 (9.8-10.0) |
| Agitation_10 ml | | | | | | | |
| Volume | | | | | | | |
| Without | 1.0 (0.8-1.2) | 1.0 (0.6-1.3) | 1.6 | 2.0 (1.6-2.4) | 1.6 (1.4-1.8) | 2.5 (2.4-2.6) | 4.6 (4.4-4.8) |
| Agitation_5 ml | | | | | | | |
| Volume | | | | | | | |
| Without | 1.3 (1.2-1.3) | 1.0 | 6.0 | 6.7 (6.5-7.0) | 6.7 (6.6-6.8) | 7.3 (7.0-7.6) | 9.4 (9.4-9.4) |
| Agitation_10 ml | | | | | | | |
| Volume | | | | | | | |

The data of Table 18 is depicted in FIG. 4. With top spray fluid bed granules of Example 4, less than 1.3 ml was aspirated after dissolving intact Naltrexone HCl tablets in various volumes of tap water (5 ml and 10 ml) at room temperature with and without agitation.

With rotor layered pellets of Example 2, the volume aspirated after dissolving intact Naltrexone HCl tablets in various volumes of tap water (5 ml and 10 ml) at room temperature with and without agitation was lower for pellets with high gelling agent content. This suggests greater gelling in water for the pellets with a high gelling agent content and low enteric coat content in comparison to the pellets with the low gelling agent content and high enteric coat content. The highest volume aspirated for the tablets containing high gelling agent content was 6 ml, and for the tablets containing low gelling agent content it was 6.9 ml.

With roller compacted and bottom spray pellets of Example 7, the volume aspirated after dissolving intact Naltrexone HCl tablets in various volumes of tap water (5 ml and 10 ml) at room temperature with and without agitation was lower for pellets with high gelling agent content. The highest volume aspirated for the tablets containing high gelling agent content was 6.7 ml, and for the tablets containing low gelling agent content it was 7.3 ml.

Roxicodone® (Oxycodone HCl tablets) was used as a comparator. The highest volume aspirated after dissolving of intact Roxicodone with tap water in room temperature was 9.9 ml. The volume aspirated from intact Roxicodone after dissolving in various volumes of tap water (5 ml and 10 ml) at room temperature with and without agitation is greater than the volume aspirated after manipulation of the tablets prepared in Examples 2, 4, and 7 under the same manipulation conditions.

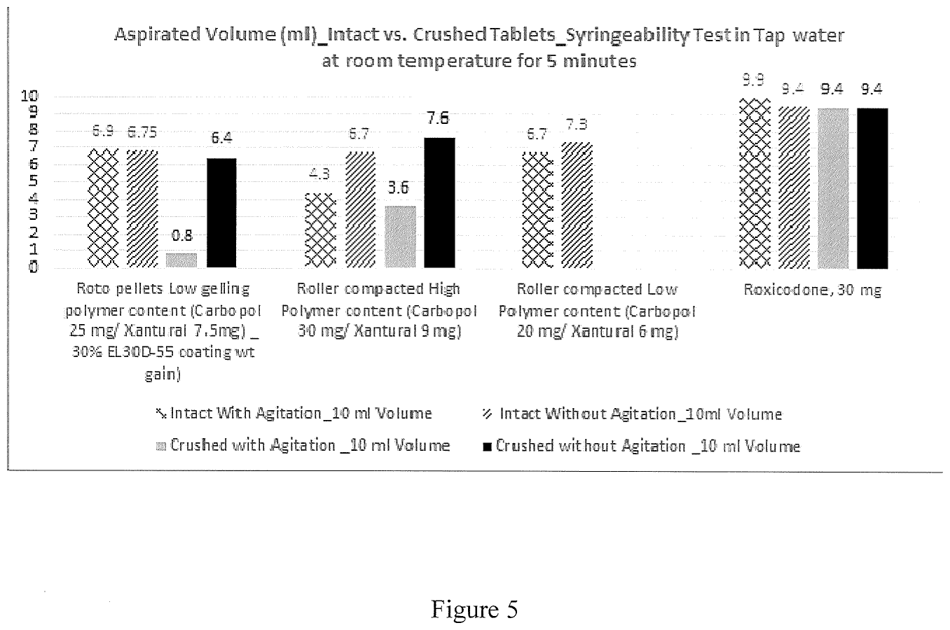

TABLE 19
Volume (ml) Aspirated After Dissolution of Tablets in Tap Water at
Room Temperature for 5 minutes (Intact vs. Crushed Tablet - FIG. 5)

| | Rotor Layering | | | |
|---|---|---|---|---|
| | Low Gelling | | | |
| | Polymer Content | Roller compaction | Roller compaction | |
| | (Carbopol 25 mg/ | High Gelling | Low Gelling | |
| | Xantural 7.5 | Polymer Content | Polymer Content | |
| | mg)_30% EL30D-55 | (Carbopol 30 mg/ | (Carbopol 20 mg/ | Roxicodone, |
| | coating wt gain) | Xantural 9 mg) | Xantural 6 mg) | 30 mg |
| | Lot No. | | | |
| | 2428-053B | 2428-069A | 2428-069B | FB0306A |
| | N = 2, Average (min-max) | | | |
| Intact with | 6.9 (6.8-7.0) | 4.3 (2.8-5.8) | 6.7 (6.4-7.0) | 9.9 (9.8-10.0) |
| Agitation_10 ml | | | | |
| Volume | | | | |
| Intact Without | 6.75 (6.5-7.0) | 6.7 (6.6-6.8) | 7.3 (7.0-7.6) | 9.4 (9.4-9.4) |
| Agitation_10 ml | | | | |
| Volume | | | | |
| Crushed with | 0.8 (0.7-0.8) | 3.6 | * | 9.4 (9.4-9.4) |
| Agitation_10 ml | | | | |
| Volume | | | | |
| Crushed without | 6.4 (6.4-6.4) | 7.6 | * | 9.4 |
| Agitation_10 ml | | | | |
| Volume | | | | |

* Data is being evaluated

The data of Table 19 is depicted in FIG. 5. With rotor layered pellets of Example 2 (low gelling agent content), the highest volume aspirated for intact tablets dissolved with 10 ml of tap water at room temperature (with and without agitation) was 6.9 ml. The volume aspirated for crushed tablets dissolved with 10 ml of tap water at room temperature was lower after agitation.

With roller compacted and bottom spray pellets of Example 7, the volume aspirated for intact and crushed tablets dissolved with 10 ml of tap water at room temperature was lower after agitation for the tablets containing high gelling agent content.

Roxicodone (Oxycodone HCl tablets) was used as a comparator. The highest volume aspirated after dissolution of intact and crushed Roxicodone with tap water in room temperature was 9.9 ml. The volume aspirated from intact and crushed Roxicodone after dissolution in tap water at room temperature with and without agitation is greater than the volume aspirated after dissolution of the tablets prepared in Examples 2 (low gelling agent content) and 7 under the same conditions.

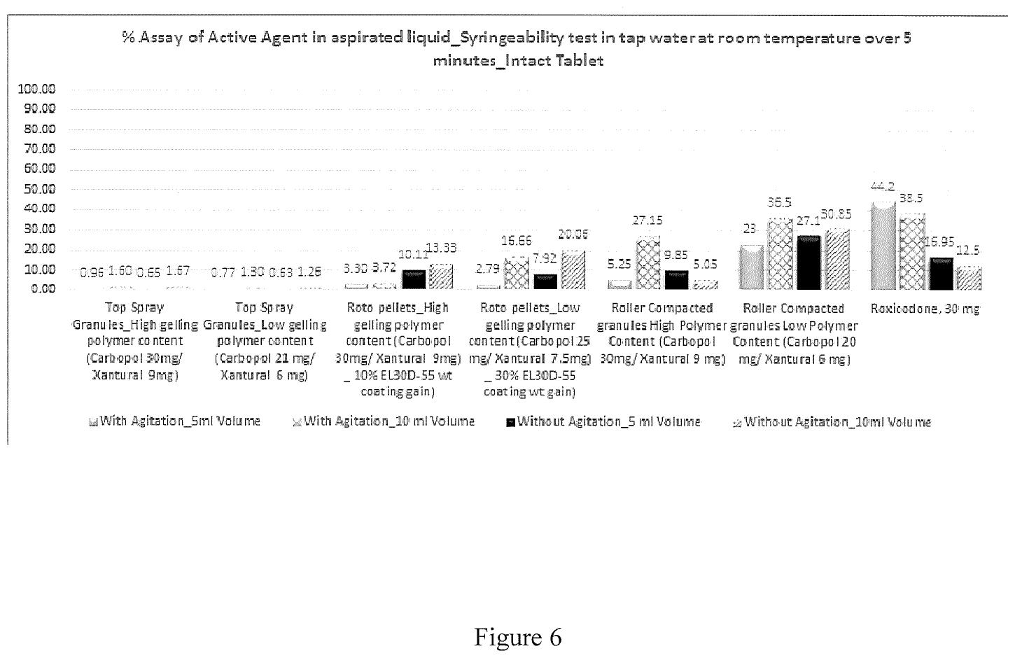

TABLE 20
Percentage Assay of Active Agent in Aspirated Liquid After Dissolution of
Intact Tablets in Tap Water at Room Temperature for 5 Minutes (FIG. 6)

| | | | Rotor Layered Pellets | | |
|---|---|---|---|---|---|
| | | | (Example 2) | | |
| | Top Spray Granules | | High Gelling | Low Gelling | |
| | (Example 4) | | Polymer Content | Polymer Content | |
| | High Gelling | Low Gelling | (Carbopol 30 mg/ | (Carbopol 25 mg/ | |
| | Polymer Content | Polymer Content | Xantural 9 mg)_{—} | Xantural 7.5 mg)_{—} | |
| | (Carbopol 30 mg/ | (Carbopol 21 mg/ | 10% EL30D-55 | 30% EL30D-55 | |
| | Xantural 9 mg) | Xantural 6 mg) | coating wt gain) | coating wt gain) | |
| | Lot No. | | | | |
| | 2428-048A | 2428-048B | 2428-053A | 2428-053B | |
| | N = 2, Average (min-max) | | | | |
| With | 0.96 (0.87-1.06) | 0.77 (0.70-0.84) | 3.30 (3.26-3.33) | 2.79 | (2.58-3.01) |
| Agitation_5 ml | | | | | |
| Volume | | | | | |
| With | 1.60 (1.46-1.73) | 1.30 (1.22-1.38) | 3.72 | 16.66 | |
| Agitation_10 ml | | | | | |
| Volume | | | | | |
| Without | 0.65 (0.60-0.70) | 0.63 (0.52-0.74) | 10.11 | 7.92 | (6.94-8.90) |
| Agitation_5 ml | | | | | |
| Volume | | | | | |
| Without | 1.67 (1.53-1.80) | 1.26 | 13.33 | 20.06 | (19.99-20.13) |
| Agitation_10 ml | | | | | |
| Volume | | | | | |

| | Roller Compacted granules | | | | | |
|---|---|---|---|---|---|---|
| | (Example 7) | | | | | |
| | High Gelling | | Low Gelling | | | |
| | Agent Content | | Agent Content | | Roxicodone, | |
| | (Carbopol 30 mg/ | | (Carbopol 20 mg/ | | 30 mg | |
| | Xantural 9 mg) | | Xantural 6 mg) | | (Control) | |
| | Lot No. | | | | | |
| | 2428-069A | | 2428-069B | | FB0306A | |
| | N = 2, Average (min-max) | | | | | |
| With | 5.25 | (4.7-5.8) | 23 | (15.5-30.5) | 44.2 | (43.6-44.8) |
| Agitation_5 ml | | | | | | |
| Volume | | | | | | |
| With | 27.15 | (16.9-37.4) | 36.5 | (36.3-36.7) | 38.5 | (37.4-39.6) |
| Agitation_10 ml | | | | | | |
| Volume | | | | | | |
| Without | 9.85 | (8.3-11.4) | 27.1 | (24.6-29.6) | 16.95 | (16.2-17.7) |
| Agitation_5 ml | | | | | | |
| Volume | | | | | | |
| Without | 5.05 | (3.2-6.91) | 30.85 | (29.9-31.8) | 12.5 | (10.2-14.8) |
| Agitation_10 ml | | | | | | |
| Volume | | | | | | |

*If a single number is listed in the table it means that a single tablet was tested

The data of Table 20 is depicted in FIG. 6. With top spray fluid bed granules of Example 4, the gelling is so pronounced that the percentage of Naloxone HCl aspirated into syringe is less than 2% of the label claim.

With Rotor Layered Pellets of Example 2, the performance in syringeability test was better for high gelling polymer content and 10% w/w of Eudragit® L30D-55 enteric coating as compared to lower gelling polymer content with 30% w/w Eudragit® L30D-55 enteric coating samples. The highest percentage of Naltrexone HCl aspirated in Rotor Layered Pellets containing high gelling agent content was 13.33% and for lower amount of gelling agent it was 20.06% of label claim, suggesting significant gelling in water to prevent syringeability of drug.

With roller compacted and bottom spray pellets, the higher gelling agent lot performed better than the lower gelling agent content lot. The highest percentage of Naltrexone HCl aspirated from the higher gelling agent lot was 27.15% and for lower amount of gelling agent it was 36.5%.

Roxicodone (Oxycodone HCl tablets) was used as a comparator. The highest percentage of Oxycodone aspirated was 44.2% of the label claim.

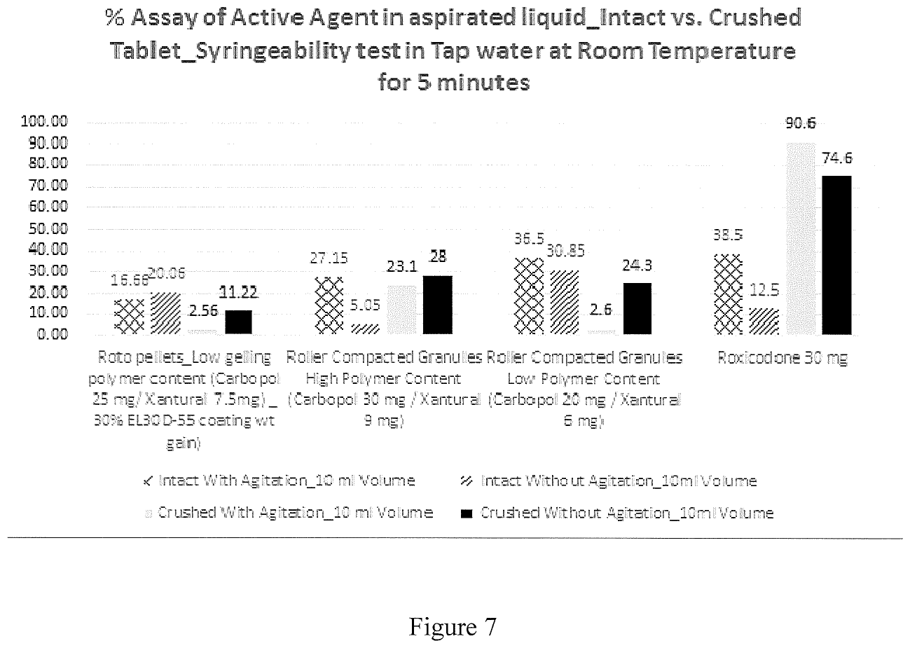

TABLE 21
Percentage Assay of Active Agent in Aspirated Liquid After Dissolution of Tablets
in Tap Water at Room Temperature for 5 Minutes (Intact vs. Crushed Tablet - FIG. 7)

| | Rotor pellets | | | | | |
|---|---|---|---|---|---|---|
| | (Example 2) | | | | | |
| | Low gelling | | Roller Compacted Granules | | | |
| | polymer content | | (Example 7) | | | |
| | (Carbopol 25 mg/ | | High Gelling | | Low Gelling | |
| | Xantural 7.5 | | Polymer Content | | Polymer Content | |
| | mg)_30% EL30D-55 | | (Carbopol 30 mg/ | | (Carbopol 20 mg/ | Roxicodone |
| | coating wt gain) | | Xantural 9 mg) | | Xantural 6 mg) | 30 mg |
| Lot No | 2428-053B | | 2428-069A | | 2428-069B | FB0306A |
| Intact with | 16.66 | | 27.15 | (16.9-37.4) | 36.5 (36.3-36.7) | 38.5 (37.4-39.6) |
| Agitation_10 ml | | | | | | |
| Volume | | | | | | |
| Intact Without | 20.06 | (19.99-20.13) | 5.05 | (3.2-6.9) | 30.85 (29.9-31.8) | 12.5 (10.2-14.8) |
| Agitation_10 ml | | | | | | |
| Volume | | | | | | |

| | | | | | |
|---|---|---|---|---|---|
| Crushed with | 2.56 | (1.78-3.33) | 23.1 | 2.6 | 90.9 (90.3-91.4) |
| Agitation_10 ml | | | | | |
| Volume | | | | | |
| Crushed Without | 11.22 | (8.43-14.02) | 28.0 | 24.3 | 74.6 |
| Agitation_10 ml | | | | | |
| Volume | | | | | |

The data of Table 21 is depicted in FIG. 7. When tablet is crushed and dissolved in water, the percent of Oxycodone syringed from Roxicodone (Oxycodone HCl Tablets) is 90.6% of the label claim whereas for sequestered gelling agent batches it is much lower.

### Example 9: Dissolution Testing

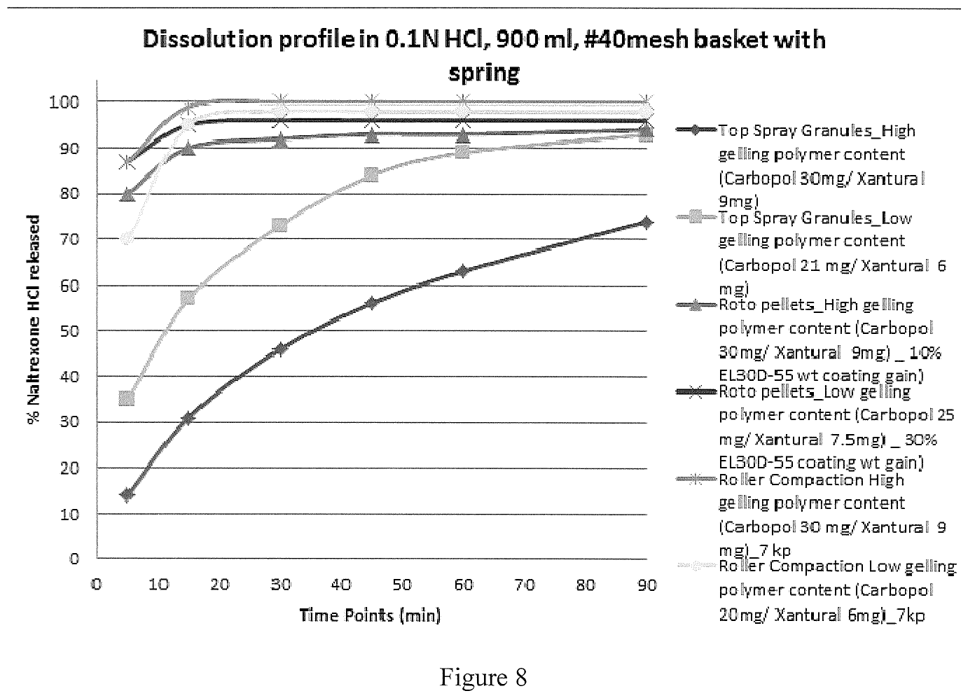

The tablets of Examples 2, 4 and 7 were tested for Active Agent release in 0.1 N HCl using basket with spring. In-vitro dissolution testing of the tablets according to Examples 9 was performed as follows: tablets were tested in vitro using a USP Apparatus 1 (#40 mesh basket) in 900 ml 0.1N HCl at 37.0±0.5° C. To reduce the propensity of the tablets, once hydrated in the dissolution medium, to stick to the solid underside of the top of the basket or the base of the shaft, a retaining spring (passivized stainless steel 316 spring, 1.5-cm outside diameter and 2-cm length) was placed in the upper part of the basket (above the tablet). Sampling time points included 5, 15, 30, 45, 60, and 90 minutes (or as indicated). Results are summarized in Tables 22 to 25 and FIGS. 8 through 11.

TABLE 22
Dissolution Profiles in 0.1N HCl, 900 ml, using Basket With Spring (FIG. 8)

| | | | Rotor pellets | | | | |
|---|---|---|---|---|---|---|---|
| | | | (Example 2) | | | | |
| | Top Spray Granules | | High Gelling | Low Gelling | Roller Compaction | | |
| | (Example 4) | | Polymer Content | Polymer Content | (Example 7) | | |
| | High Gelling | Low Gelling | Carbopol 30 mg/ | Carbopol 25 mg/ | High Gelling | | Low Gelling |
| | Polymer Content | Polymer Content | Xantural 9 mg) | Xantural 7.5 mg) | Polymer Content | | Polymer Content |
| | Carbopol 30 mg/ | Carbopol 21 mg/ | 10% EL30D-55 | 30% EL30D-55 | Carbopol 30 mg/ | | Carbopol 20 mg/ |
| | Xantural 9 mg | Xantural 6 mg | coating wt gain | coating wt gain | Xantural 9 mg | | Xantural 6 mg |
| | Lot No. | | | | | | |
| Time (mins) | 2428-048A | 2428-048B | 2428-053A | 2428-053B | 2428-069A | | 2428-069B |
| 5 | 14 (12-16) | 35 (28-42) | 80 (71-92) | 87 (86-88) | 87 | (85-88) | 70 (57-85) |
| 15 | 31 (29-34) | 57 (47-63) | 90 (85-94) | 95 (94-97) | 99 | (97-101) | 95 (92-96) |
| 30 | 46 (43-48) | 73 (63-85) | 92 (89-94) | 96 (94-97) | 100 | (97-102) | 98 (96-99) |
| 45 | 56 (51-60) | 84 (78-94) | 93 (91-94) | 96 (94-97) | 100 | (98-102) | 98 (96-99) |
| 60 | 63 (57-68) | 89 (82-97) | 93 (92-94) | 96 (94-98) | 100 | (98-102) | 98 (96-99) |
| 90 | 74 (65-80) | 93 (88-97 ) | 94 (93-94) | 96 (94-98) | 100 | (98-102) | 98 (96-99) |

The data of Table 22 is depicted in FIG. 8. Dissolution profile of tablets made with top spray fluid bed granulation approach (Example 4) was slower than the tablets made with rotor layering (Example 2) and roller compaction approach (Example 7).

For tablets made using sequestered gelling agents by rotor layering approach (Example 2) and by roller compaction approach (Example 7), 90% or more drug release was achieved in 15 minutes suggesting that the drug release is not impacted in acidic media as the gelling agents are coated with Eudragit® L30D-55 (a polymer that dissolves at pH higher than 5.5).

TABLE 23
Effect of Hardness on Dissolution Profiles of Tablets Made With Top Sprayed Gelling
Agent Granules (Example 4) in 0.1N HCl, 900 mL, using Basket With Spring

| | Time (mins) | | | |
|---|---|---|---|---|
| | Top Spray | Top Spray | Top Spray | Top Spray |
| | Granules | Granules | Granules | Granules |
| | (Carbopol 30 mg/ | (Carbopol 30 mg/ | (Carbopol 21 mg/ | (Carbopol 21 mg/ |
| | Xantural 9 mg) | Xantural 9 mg) | Xantural 6 mg) | Xantural 6 mg) |
| | Lot | | | |
| | 2428-048A | 2428-048A | 2428-048B | 2428-048B |
| | Hardness | | | |
| Time (mins) | 10 kp | 17 kp | 10 kp | 17 kp |
| 5 | 14 (12-16) | 15 (14-16) | 35 (28-42) | 34 (30-38) |
| 15 | 31 (29-34) | 41 (38-45) | 57 (47-63) | 67 (54-73) |
| 30 | 46 (43-48) | 60 (53-66) | 73 (63-85) | 82 (69-90) |
| 45 | 56 (51-60) | 69 (62-76) | 84 (78-94) | 88 (76-95) |
| 60 | 63 (57-68) | 75 (67-81) | 89 (82-97) | 91 (81-97) |
| 90 | 74 (65-80) | 82 (75-88) | 93 (88-97) | 94 (86-98) |

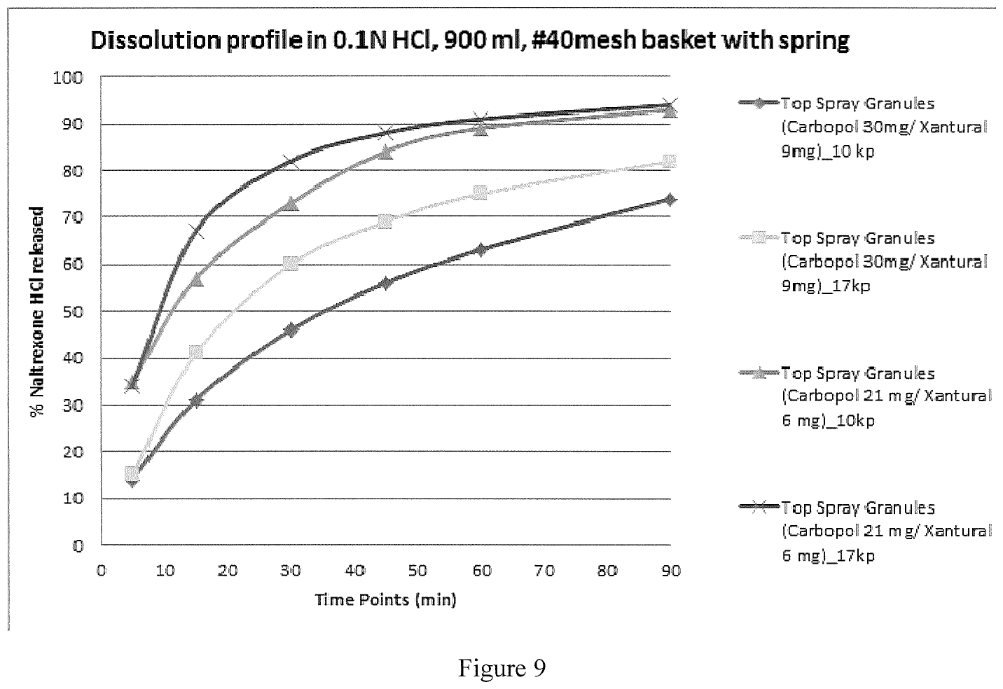

The data of Table 23 is depicted in FIG. 9. Dissolution profile of tablets made with top spray fluid bed granulation approach (Example 4) was slower for tablets with a hardness value of 10 kP than for tablets with a hardness value of 17 kP. Additionally, the dissolution profile for tablets from Example 4 with low amounts of gelling agents were slower than for tablets from Example 4 with high amounts of gelling agents.

TABLE 24
Effect of Hardness on Dissolution Profiles of Tablets
Made With Rotor Layered Gelling Agent Pellets (Example
2) in 0.1N HCl, 900 mL, using Basket With Spring

Time (mins)

| | |
|---|---|
| Rotor Pellets | Rotor Pellets |
| (Carbopol 25 mg/ | (Carbopol 25 mg/ |
| Xantural 7.5 mg) | Xantural 7.5 mg) |
| 30% EL30D-55 | 30% EL30D-55 |
| coating wt gain) | coating wt gain) |

Lot No.

| | |
|---|---|
| 2428-053B | 2428-053B |

Hardness

| Time (mins) | 5 kp | 8 kp |
|---|---|---|
| 5 | 87 (86-88) | 33 (32-35) |
| 15 | 95 (94-97) | 91 (91-92) |
| 30 | 96 (94-97) | 93 (93-94) |
| 45 | 96 (94-97) | 93 (93-94) |
| 60 | 96 (94-98) | 93 (93-94) |
| 90 | 96 (94-98) | 93 (93-94) |

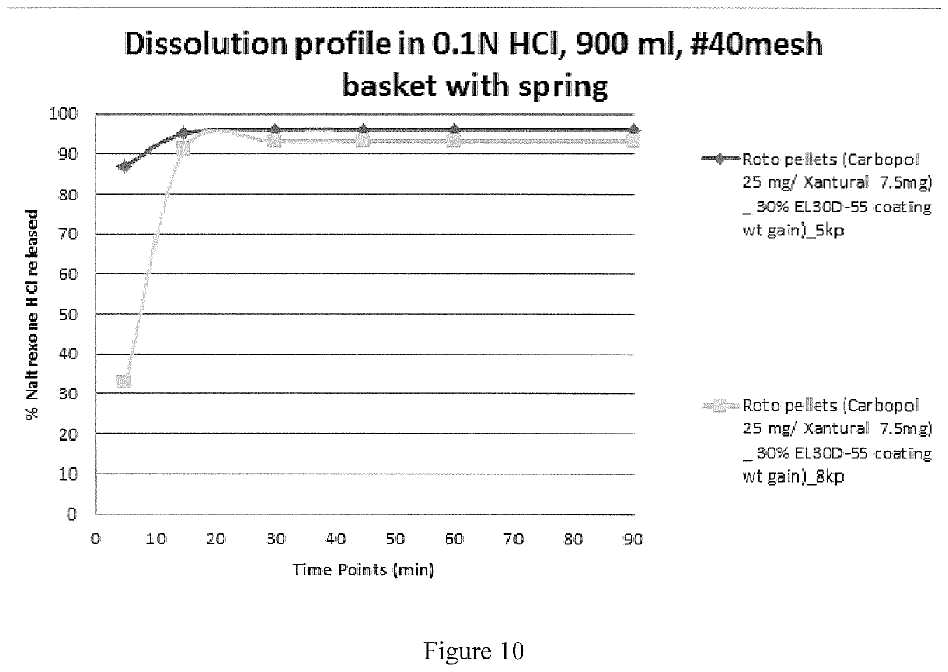

The data of Table 24 is depicted in FIG. 10. Dissolution profile of tablets made with Rotor Layered Gelling Agent Pellets approach (Example 2) was similar regardless of the hardness.

TABLE 25
Effect of Hardness on Dissolution profile of Tablets Made With Roller
Compacted, Wurster Coated Granules (Example 7) of Gelling Polymers

| | Time (mins) | | | | |
|---|---|---|---|---|---|
| | Roller Compaction | | Roller Compaction | Roller Compaction | Roller Compaction |
| | High Gelling | | High Gelling | Low Gelling | Low Gelling |
| | Agent Content | | Agent Content | Agent Content | Agent Content |
| | (Carbopol 30 mg/ | | (Carbopol 30 mg/ | (Carbopol 20 mg/ | (Carbopol 20 mg/ |
| | Xantural 9 mg) | | Xantural 9 mg) | Xantural 6 mg) | Xantural 6 mg) |
| | Lot No. | | | | |
| | 2428-069A | | 2428-069A | 2428-069B | 2428-069B |
| | Hardness | | | | |
| Time (mins) | 7 kp | | 15 kp | 7 kp | 15 kp |
| 5 | 87 | (85-88) | 87 (84-92) | 70 (57-85) | 84 (77-89) |
| 15 | 99 | (97-101) | 98 (97-99) | 95 (92-96) | 97 (95-98) |
| 30 | 100 | (97-102) | 98 (97-99) | 98 (96-99) | 98 (95-99) |
| 45 | 100 | (98-102) | 98 (97-99) | 98 (96-99) | 98 (96-99) |
| 60 | 100 | (98-102) | 99 (97-99) | 98 (96-99) | 98 (96-99) |
| 90 | 100 | (98-102) | 99 (97-100) | 98 (96-99) | 98 (96-99) |

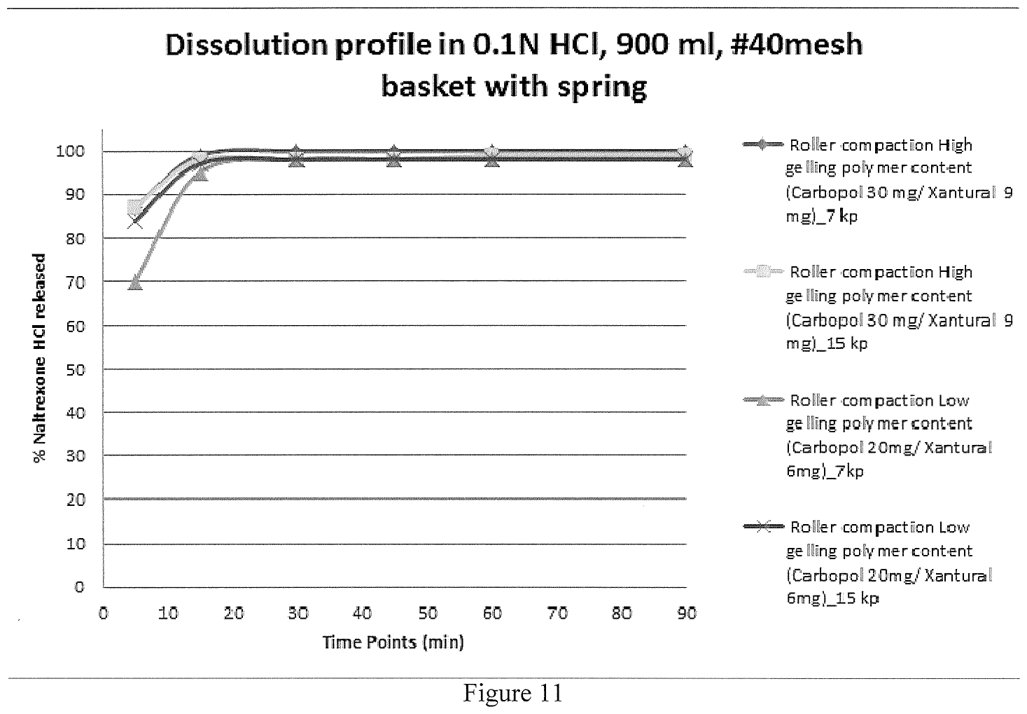

The data of Table 25 is depicted in FIG. 11. Dissolution profile of tablets with roller compacted and Wurster coated granules of gelling agent (Example 7) was similar regardless of the hardness and regardless of content of gelling agent.

### Summary of Examples 8-9

TABLE 26
Summary of Examples 8-9 and Tables 18-25

| | Maximum % of Active Agent aspirated in | | | |
|---|---|---|---|---|
| | syringe in 10 mL tap water at room | | | % Naltrexone HCl |
| | temperature for 5 minutes | | | Dissolved in |
| | | Intact | Crushed | 15 minutes |
| Top Spray Fluid bed | High polymer | 1.67 | | 41 |
| granules in tablet | content | | | |
| | Low polymer | 1.30 | | 67 |
| | content | | | |
| Rotor-layered pellets | High polymer | 13.33 | | 90 |
| in tablet | content | | | |
| | Low polymer | 20.06 | 11.22 | 95 |
| | content | | | |
| Roller compacted | High polymer | 27.15 | 28.00 | 99 |
| bottom spray fluid | content | | | |
| bed coated granules | | | | |
| in tablet | | | | |
| Roxicodone | | 44.20 | 90.30 | |
| (marketed IR | | | | |
| Oxycodone product) | | | | |

Coating of gelling agent with Eudragit® L30D-55 and incorporating in an immediate release dosage form affords an immediate release of drug from the dosage form in acidic pH (<pH 5.5) found in stomach. When the tablet is dissolved in water (up to 10mL) for injection for IV abuse, the Eudragit coating dissolves in water having pH greater than 5.5. Thus the gelling agents would be released and make the solution viscous preventing syringeability of drug being abused. All three techniques used to cover gelling agents with Eudragit® L30D-55 show reduction in the amount of drug that can be syringed upon dissolving in water. With top spray fluid bed granulation approach, the dissolution profile of drug was slower.

Incorporation of Sodium Lauryl Sulfate, which is a nasal irritant, into the tablets of examples 2, 4, and 7 prevents nasal abuse of an opioid formulation in addition to preventing abuse through Intravenous route with the use of sequestered gelling agents.

For simplicity of explanation, the embodiments of the methods of this disclosure are depicted and described as a series of acts. However, acts in accordance with this disclosure can occur in various orders and/or concurrently, and with other acts not presented and described herein. Furthermore, not all illustrated acts may be required to implement the methods in accordance with the disclosed subject matter. In addition, those skilled in the art will understand and appreciate that the methods could alternatively be represented as a series of interrelated states via a state diagram or events.

In the foregoing description, numerous specific details are set forth, such as specific materials, dimensions, processes parameters, etc., to provide a thorough understanding of the present invention. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. Reference throughout this specification to “an embodiment”, “certain embodiments”, or “one embodiment” means that a particular feature, structure, or characteristic described in connection with the embodiment is included in at least one embodiment. Thus, the appearances of the phrase “an embodiment”, “certain embodiments”, or “one embodiment” in various places throughout this specification are not necessarily all referring to the same embodiment.

The present invention has been described with reference to specific exemplary embodiments thereof. The specification and drawings are, accordingly, to be regarded in an illustrative rather than a restrictive sense. Various modifications of the invention in addition to those shown and described herein will become apparent to those skilled in the art and are intended to fall within the scope of the appended claims.

1. A solid oral dosage form comprising an opioid analgesic composition in an immediate release form and a gelling agent composition in a delayed release form, wherein the solid oral dosage form is free of or substantially free of the opioid analgesic composition in an extended release form.

2. The solid oral dosage form of claim 1, wherein the opioid analgesic is selected from the group consisting of morphine, hydromorphone, hydrocodone, oxycodone, codeine, levorphanol, meperidine, dihydrocodeine, dihydromorphine, oxymorphone, fentanyl, buprenorphine pharmaceutically acceptable salts thereof, solvates thereof, prodrugs thereof, and mixtures thereof.

3. (canceled)

4. The solid oral dosage form of claim 1 comprising from about 0.1% to about 80% (w/w), or from about 0.5% to about 30% (w/w), or from about 1% to about 10% (w/w) opioid analgesic.

5. The solid oral dosage form of claim 1, wherein the solid oral dosage form releases at least about 80%, or at least about 85%, or at least about 90%, or at least about 95% of the opioid analgesic within 30 minutes as measured by in-vitro dissolution in a USP Apparatus 1 (#40 mesh basket) in 900 ml 0. IN HCl at room temperature.

6-7. (canceled)

8. The solid oral dosage form of claim 1, wherein the delayed release gelling agent composition comprises a gelling agent and an enteric material.

9. The solid oral dosage form of claim 8, wherein the gelling agent is selected from the group consisting of starch, starch derivatives, sodium carboxymethylcellulose, methylcellulose, hydroxyethylcellulose, hydroxypropylcellulose, hydroxypropylmethylcellulose, attapulgites, bentonites, dextrins, alginates, carrageenan, gum tragacanth, gum acacia, guar gum, xanthan gum, pectin, gelatin, kaolin, cross linked polyacrylic acid, polyvinylpyrrolidone, polyethylene oxide, polyvinyl alcohol, and mixtures thereof.

10. The solid oral dosage form of claim 8, wherein the gelling agent is selected from the group consisting of polyethylene oxide, xanthan gum, cross linked polyacrylic acid, polysaccharides, and mixtures thereof.

11-17. (canceled)

18. The solid oral dosage form of claim 8, wherein the gelling agent comprises xanthan gum and carbomer homopolymer.

19. The solid oral dosage form of claim 1, comprising from about 0.1% to about 50%, or from about 0.5% to about 20%, or from about 1% to about 10% gelling agent (w/w).

20. The solid oral dosage form of claim 1, wherein the viscosity of a solution obtained from an intact solid oral dosage form at 5 minutes in about 0.5 ml to about 10 ml water at room temperature is about 50 cP or more, about 75 cP or more, about 100 cP or more, or about 125 cP or more, wherein the viscosity is measured by a rotational viscometer.

21-24. (canceled)

25. The solid oral dosage form of claim 1, wherein the ratio of the viscosity of a solution obtained from an intact solid oral dosage form at 5 minutes in about 5 ml water at room temperature to the viscosity of a solution obtained from an intact solid oral dosage form at 5 minutes in about 5 ml 0. IN HCl at room temperature is about 10:1 or more, about 15:1 or more, about 20:1 or more, about 25:1 or more, or about 30:1 or more wherein the viscosity is measured by a rotational viscometer.

26. The solid oral dosage form of claim 1, wherein the opioid analgesic composition is in the form of one or more particles.

27. (canceled)

28. The solid oral dosage form of claim 8, wherein the delayed release gelling agent composition is in the form of one or more particles.

29. The solid oral dosage form of claim 28, wherein each delayed release gelling agent particle comprises (i) the gelling agent coated with the enteric material, (ii) an inert core coated with the gelling agent and overcoated with the enteric material, or (iii) the gelling agent dispersed in matrix material.

30. The solid oral dosage form of claim 26, wherein the one or more opioid analgesic particles and the one or more delayed release gelling agent particles are contained in a pharmaceutically acceptable capsule.

31. (canceled)

32. The solid oral dosage form of claim 28, wherein the opioid analgesic composition is coated on the one or more delayed release gelling agent particles.

33-35. (canceled)

36. The solid oral dosage form of claim 8, wherein the enteric material dissolves above a pH of about 5.5 and does not dissolve below a pH of about 5.5.

37. (canceled)

38. The solid oral dosage form of claim 8, wherein the enteric material is selected from the group consisting of methacrylic acid/methyl methacrylate, methacrylic acid/ethyl acrylate copolymers, methacrylic acid/methyl acrylate/methyl methacrylate copolymers, shellac, hydroxypropyl methylcellulose phthalate, hydroxyl propyl methyl cellulose acetate succinate, hydroxypropyl methyl cellulose trimellitate, cellulose acetate phthalates, polyvinyl acetate phthalates and mixtures thereof.

39-109. (canceled)

110. A method of treating pain comprising administering a solid oral dosage form of claim 1.

111-115. (canceled)

116. A process for preparing a solid oral dosage form comprising (i) preparing one or more particles; (ii) coating the one or more particles with an opioid analgesic composition; and (iii) blending the one or more particles coated with opioid analgesic composition with a delayed release gelling agent composition, wherein the solid oral dosage form comprises an opioid analgesic composition in an immediate release form, and wherein the solid oral dosage form is free of or substantially free of an opioid analgesic composition in an extended release form.

117-120. (canceled)
